(19) United States
(12) Patent Application Publication
(10) Pub. No.: US 2020/0239824 A1
(43) Pub. Date: Jul. 30, 2020

(54) Hydrogel for stem cell and organoid culture

(71) Applicant: TheWell Bioscience, Newark, NJ (US)

(72) Inventor: Hongzhou HUANG, North Brunswick, NJ (US)

(21) Appl. No.: 16/834,302

(22) Filed: Mar. 30, 2020

Related U.S. Application Data
(63) Continuation-in-part of application No. 15/949,457, filed on Apr. 10, 2018, now Pat. No. 10,603,406.
(60) Provisional application No. 62/483,831, filed on Apr. 10, 2017.

(51) Int. Cl. C12M 1/12; C12M 3/00; C12N 5/074; A61K 47/69; A61K 47/36; A61K 35/545; A61K 9/00

(52) U.S. Cl. CPC C12M 25/14; C12N 2501/12; C12N 5/0607; A61K 47/6903; A61K 47/36; A61K 35/545; A61K 9/0019; C12N 2513/00; C12N 2501/11; C12N 2501/105; C12N 2501/155; C12N 2501/13; C12N 2501/41; C12N 2501/315; C12N 2501/16; C12N 2501/998; C12N 2533/52; C12N 2533/50; C12N 2501/999; C12N 2501/113; C12N 2501/115; C12M 21/08

(57) ABSTRACT

Methods using a soft polysaccharide hydrogel for an organoid culture are described. An example method includes preparing a cell suspension in a cell culture medium. The cells of the cell suspension embedded or cultured in the 3D cell culture biomatrix are injectable for in vivo application. The method also includes mixing a hydrogel solution with the cell suspension to form a soft hydrogel mixture, adding additional cell culture medium to the soft hydrogel mixture to obtain cell colonies after a first time period, harvesting the cell colonies, mixing the hydrogel solution with the cell colonies to create a 3D cell culture biomatrix, adding cell differential medium to the hydrogel solution, replacing the cell differential medium with an organoid transfer medium after a second time period, and replacing the organoid transfer medium with an organoid medium after a third time period.

### CROSS-REFERENCE TO RELATED APPLICATIONS

This application is a U.S. Continuation-in-Part Patent Application that claims priority to U.S. Non-Provisional patent application Ser. No. 15/949,457 filed on Apr. 10, 2018, which claims priority from U.S. Provisional Application Ser. 62/483,831 filed on Apr. 10, 2017, the entire contents of which are hereby incorporated by reference.

### FIELD OF THE EMBODIMENTS

The field of the embodiments of the present invention relate to the use of hydrogels for cell cultures and various other biomedical applications. More specifically, the embodiments of the present invention relate to use of biocompatible hydrogels in a stem cell and organoid culture, having in vivo application.

### BACKGROUND OF THE EMBODIMENTS

For years, the examination of biological phenomenon has been routinely explored two-dimensional (2D) environments on unphysiologically rigid materials, such as glass coverslips. This resulted in a misunderstanding of many cellular behaviors, such as cell shape, proliferation, and differentiation. These 2D systems also prevented the proper examination of cellular responses to extracellular cues. For example, in 2D cultures, cells sense their surroundings via dynamic leading-edge filopodia (A. Arjonen, R. Kaukonen, and J. Ivaska, “Filopodia and adhesion in cancer cell motility,” Cell Adhesion and Migration, 2011).

However, it is now well known that cells also possess actin-rich apical and basal protrusions, such as invadopodia and podosomes, to sense and respond to the environment in 3D (M. Gimona, R. Buccione, S. A. Courtneidge, and S. Linder, “Assembly and biological role of podosomes and invadopodia,” Current Opinion in Cell Biology, 2008; D. A. Murphy and S. A. Courtneidge, “The ‘ins’ and ‘outs’ of podosomes and invadopodia: Characteristics, formation and function,” Nature Reviews Molecular Cell Biology, 2011; and C. M. Gould and S. A. Courtneidge, “Regulation of invadopodia by the tumor microenvironment,” Cell Adhesion and Migration, 2014). Invadopodia are critical regulators of extracellular matrix (ECM) remodeling during cancer metastasis, illustrating the importance of studying cellular behaviors in 3D.

Three-dimensional (3D) cell culture models are a more accurate representation of the natural environment experienced by the cells in the living organism, which allows for intercellular interactions with more realistic biochemical and physiological responses. In 3D cell cultures, cells behave and respond more like they would in vivo to internal and external stimuli, such as changes in temperature, pH, nutrient absorption, transport, and differentiation. Therefore, scientists are shifting their focus from 2D to 3D cell cultures in the fields of drug screening, tissue engineering, preclinical study, cell therapy, and basic cell biological study.

To mimic in vivo cell growing conditions, the reticulated structure of 3D scaffold should be serialized, have a high water content, and have a number of other desirable characteristics. Such other desirable characteristics include accurate 3D spatial support, suitable mechanical strength, and facile transportation of oxygen, nutrients, waste, and soluble factors. Mild and cytocompatible conditions for sol-gel transformation are preferred, to ensure that cells survive comfortably during both cell encapsulation and isolation. Moreover, the injectable property of biomaterials used for 3D cell cultures is critical for downstream applications, which includes cancer therapy (xerography study for drug discovery), tissue regeneration, and 3D bio-printing.

The current materials for 3D cell cultures on the market can be classified as hydrogels, polymer matrices, hanging drop plates, low adhesion plates, micro-patterned surfaces, and magnetic levitations. The development of the 3D cell culture technology, organoids (or self-organized three-dimensional tissue cultures) has opened a new way for diseases to be studied and treated. The 3D assembling organoids contain multiple cell types, arranged similarly to the cells in a specific tissue. Organoids made from human cells or patient tissues become a valuable tool for medical research, and specifically for preclinical studies. To culture organoids, the hydrogel biomaterial plays a critical role.

Hydrogel scaffolds have been demonstrated as the most promising approach to date in facilitating 3D cell culture. Hydrogels allow experimenters to encapsulate cells within the hydrogels or seed cells post-formation (S. R. Caliari and J. A. Burdick, “A practical guide to hydrogels for cell culture,” Nature Methods, 2016). Organic microenvironments, e.g. collagen, expose cells to native culture systems possessing a variety of functional ligands (K. Wolf et al., “Collagen-based cell migration models in vitro and in vivo,” Seminars in Cell and Developmental Biology, 2009). Providing cells with a tissue-like environment permits the examination of unique cellular characteristics dependent upon expression of specific cell surface receptors. However, while organic hydrogels produce a cell culture system similar to in vivo conditions, there are drawbacks (S. R. Caliari and J. A. Burdick, “A practical guide to hydrogels for cell culture,” Nature Methods, 2016).

Most existing biomaterials (including hydrogel scaffolds) for 3D cell cultures are limited to physiological conditions (e.g. poor scaffold structure, unwanted growth factors, and undesirable pH or temperature of pre-gel solution), complex operating steps for cell encapsulation, difficulties for cell isolation from the culture scaffold, and limited product reproducibility. In addition, injectable properties, such as shear-thinning and rapid recovery of physical strength, in currently marketed hydrogel materials is very rare. This drawback not only affects the data generated from these 3D cell culture technologies, but also limits the applications of this technology for downstream analysis and clinical applications.

Further, using native ECM in a cell culture is restrictive due to inconsistencies during production, presence of growth factors, and inconsistencies of ligand bioactivity. To address these issues, more robust synthetic hydrogel systems are needed (S. L. Bellis, “Advantages of RGD peptides for directing cell association with biomaterials,” Biomaterials, 2011; T. Maeda, K. Titani, and K. Sekiguchi, “Cell-adhesive activity and receptor-binding specificity of the laminin-derived YIGSR sequence grafted onto Staphylococcal protein a,” J. Biochem., 1994; and T. Y. Cheng, M. H. Chen, W. H. Chang, M. Y. Huang, and T. W. Wang, “Neural stem cells encapsulated in a functionalized self-assembling peptide hydrogel for brain tissue engineering,” Biomaterials, 2013).

Synthetic hydrogels permit greater reliability and control of experimental conditions. With greater hydrogel reproducibility, scientists can be assured observed cellular behaviors and characteristics are consistent and accurate. Thus, a need exists for methods using hydrogels in an organoid culture.

Examples of related art are described below:

U.S. Patent Application No. 2008/0220526 pertains to coatings for cell culture surfaces. More particularly, this invention relates to coatings for cell culture surfaces which are derived from or contain gums including naturally occurring gums, plant gums, galactomannan gums or derivatives thereof. The invention also relates to articles of manufacture (e.g., cell culture vessels and labware) having such coatings, methods of applying these coatings to cell culture surfaces, and methods of using coated cell culture vessel.

U.S. Pat. No. 9,579,417 pertains to cell-adhesive gellan gum spongy-like hydrogels that are able to entrap/encapsulate adherent cells, which spread within the material, maintaining their phenotype and remaining viable and proliferative. The methodology used to obtain these materials involves hydrogel preparation, freezing, freeze-drying and re-hydration with a saline solution with cells and with/without bioactive molecules. No pre and/or post functionalization of the spongy-like hydrogels with cell adhesive features, as used for other hydrogels, is used. The cell adhesive character of these materials, not observed in hydrogels, is in part explained by their physical properties, between sponges and hydrogels, dissimilar from the precursor hydrogels. The physical properties that are mainly different are the morphology, microstructure, water content, and mechanical performance. Gellan gum spongy-like hydrogels physical properties and biological performance can be tuned by manipulating the parameters involved in spongy-like hydrogel formation. Bioactive molecules can also be entrapped with or without cells to modify the biological performance of the spongy-like hydrogels. These materials can be applied in the context of bioengineering, tissue engineering, regenerative medicine and biomedical applications.

Chinese Patent Application No. 106474560 pertains to the technical field of biological material, discloses for 3D biological printing of the hydrogel material and its preparation method and application. Hydrogel material of this invention comprises the following mass percentage component: and/or its derivatives to form the 0.5-10%, PEG and/or its derivatives 0.1-20%, cross-linking initiator 0-1%, biological active component 0-15%, the rest of the solvent. The invention is based on the form the hydrogel material and PEG double-network hydrogel, physiological environment forming an interpenetrating double-network structure, has better structure and size stability, has fast gel under physiological conditions, cell with good biocompatibility, immune rejection small, cell encapsulation rate high, the mechanical strength of the controllable, biodegradable and the like. And applied to the 3D in biological printing, overcomes the slow curing speed, curing conditions are harsh, mechanical property is limited, cells poor compatibility, has obvious advantages and good industrialization prospects.

PCT Patent Application No. WO 2014/025312 pertains to a method of manufacturing hydrogel microparticles comprising one or more species of living cells attached thereon and/or encapsulated therein is provided. The method includes dissolving a hydrogel-forming agent in an aqueous medium to form a solution; suspending one or more species of living cells in the solution to form a cell suspension; dispersing the cell suspension into an organic oil to form a microemulsion; and subjecting the microemulsion to conditions that allow the hydrogel-forming agent to form hydrogel microparticles comprising one or more species of living cells attached thereon and/or encapsulated therein. Composition comprising a mixture of a degradable hydrogel and at least one hydrogel microparticle having one or more species of living cells, and method of manufacturing a scaffold for tissue engineering are also provided.

PCT Patent Application No. 2014/017513 pertains to a method for culturing a cell and/or a tissue, said method being characterized by culturing the cell and/or the tissue in a floated state using a culture medium composition, wherein amorphous structures are formed in a liquid culture medium, are dispersed in the solution uniformly, and substantially hold the cell and/or the tissue without substantially increasing the viscosity of the solution, so that the culture medium composition has an effect of preventing the sedimentation of the structures; and others.

Various methods are known in the art. However, their means of operation are substantially different from the present disclosure, as the other inventions fail to solve all the problems taught by the present disclosure. The present invention and its embodiments relate to the use of hydrogels for cell cultures and various other biomedical applications. More specifically, the embodiments of the present invention relate to use of biocompatible hydrogels in a stem cell and organoid culture, having in vivo application.

### SUMMARY OF THE EMBODIMENTS

The present invention describes use of hydrogels for cell cultures and various other biomedical applications. More specifically, the embodiments of the present invention relate to use of hydrogels in an organoid culture.

A first embodiment of the instant invention describes a method using a soft polysaccharide hydrogel for an organoid culture. The method includes preparing a cell suspension in a cell culture medium. The cell suspension includes single stem cells, stem cell clusters, cells from an isolated tissue, cells from a xenograft sample, and/or cells from an organoid fragment, among other cells not explicitly listed herein. As described herein, a “xenograft” is a tissue graft or organ transplant from a donor of a different species from the recipient. Regarding the cells from the xenograft sample, cells may first be isolated from patient tissue and then the cells may be injected to an animal (xenograft). The cells may then be harvested from the animal to achieve a higher cell number or more mature cells. The harvested cells may then be induced for the organoid.

In some examples, the cell culture medium includes a growth factor/protein and an inhibitor/small molecule to induce the cells to the organoid culture. The growth factor/protein includes an epidermal growth factor (EGF), an insulin-like growth factor (IGF), a fibroblast growth factor (FGF), R-Spondin, Wnt-3a, a bone morphogenetic protein (BMP), a hepatocyte growth factor, Activin A, a dickkopf-related protein, a brain-derived neurotrophic factor, a glial cell-derived neurotrophic factor, sonic hedgehog, heregullin, prolactin, and/or Noggin, among others. The inhibitor/small molecule includes ROCK1, Thiazovicin, CHIR99021, LY294002, A 83-01, Nicotinamide, SB 202190, Gastrin, DAPT, Forskolin, Prostaglandin E2, Testisteribe, SB 431542, Retinoic acid, Y27632, MD2206, Dorsomorphin, G27632, and/or a smoothened agonist, among others.

The method also includes mixing a hydrogel solution with the cell suspension to form a soft hydrogel mixture. An elastic modulus of the hydrogel solution is in a range between approximately 0.001 Pa to approximately 5000 Pa. The elastic modulus of the soft hydrogel mixture is less than 50 Pa when the cell colony needs to be formed quickly and then the organoid inducement is started from the cell colony. In other examples, the cells may be encapsulated in the hydrogel biomatrix (having an elastic modulus between 1 Pa to 5000 Pa) and then a medium is added to the hydrogel biomatrix to induce the organoid directly.

In some examples, an inhibitor/small molecule and/or a growth factor may be added to the soft hydrogel mixture. The inhibitor/small molecule may include: ROCK1, Thiazovicin, CHIR99021, LY294002, A 83-01, Nicotinamide, SB 202190, Gastrin, DAPT, Forskolin, Prostaglandin E2, Testisteribe, SB 431542, retinoic acid, Y27632, MD2206, Dorsomorphin, G27632, and/or the smoothened agonist, among others. The growth factor may include: the EGF, the IGF, the FGF, R-Spondin, Wnt-3a, the BMP, the hepatocyte growth factor, Activin A, the dickkopf-related protein, the brain-derived neurotrophic factor, the glial cell-derived neurotrophic factor, the sonic hedgehog, heregullin, prolactin, and/or Noggin, among others.

In some examples, the method may include modifying the hydrogel solution with a functional ligand and/or a functional peptide. The functional ligand includes RGD, a matrix metallopeptidase (MMP) sensitive ligand, a laminin functional ligand, a vitronectin functional ligand, a fibronectin functional ligand, an osteopontin functional ligand, a nidogen functional ligand, an elastin functional ligand, a thrombospondin functional ligand, and/or a collagen functional ligand, among others. The functional peptide includes a MMP functional peptide, a collagen functional peptide, a vitronectin functional peptide, a laminin functional peptide, and/or a functional peptide molecule having an amine group, a carboxyl group, and an amide group, among others. The functional peptide molecule having an amine group, the carboxyl group, and the amide group includes RGD, IKVAV, REDV, YIGSRY, and/or poly Lysin.

The method further includes adding additional cell culture medium to the soft hydrogel mixture to obtain cell colonies after a first time period in a range of approximately one day to approximately seven days for growth of the cell colonies to become a size in a range of approximately 10 micrometers to approximately 1000 micrometers in diameter. Next, the method includes harvesting the cell colonies and mixing the hydrogel solution with the cell colonies to create a three-dimensional (3D) cell culture biomatrix. The cells of the cell suspension embedded or cultured in the 3D cell culture biomatrix are injectable for in vivo application.

The method may further include adding an inhibitor/small molecule and/or a growth factor to the cell culture biomatrix. The inhibitor/small molecule includes: ROCK1, Thiazovicin, CHIR99021, LY294002, A 83-01, Nicotinamide, SB 202190, Gastrin, DAPT, Forskolin, Prostaglandin E2, Testisteribe, SB 431542, Retinoic acid, Y27632, MD2206, Dorsomorphin, G27632, and/or the smoothened agonist, among others. The growth factor includes: the EGF, the IGF, the FGF, R-Spondin, Wnt-3a, the BMP, the hepatocyte growth factor, Activin A, the dickkopf-related protein, the brain-derived neurotrophic factor, the glial cell-derived neurotrophic factor, sonic hedgehog, heregullin, prolactin, and/or Noggin, among others.

The method further includes adding cell differential medium to the hydrogel solution. The cell differential medium comprises activin A, the FGF, and/or the IGF, among other materials not explicitly listed herein. After a second time period in a range of approximately one day to approximately seven days, the method includes replacing the cell differential medium with an organoid transfer medium. The organoid transfer medium comprises activin A, the FGF, Noggin, the EGF, and/or R-spondin-1, among other materials not explicitly listed herein. Moreover, after a third time period in a range of approximately twenty-four hours to approximately forty-eight hours, the method includes replacing the organoid transfer medium with an organoid medium. The organoid medium comprises Noggin, the EGF, and/or R-spondin-1, among other materials not explicitly listed herein.

In examples, a universal medium may be used to induce the cells directly to the organoid. In other examples, the medium may be reformulated to directly induce the single cells or the cell colonies to the organoid. In some examples, use of the cell differential medium is not needed and one may use of the organoid transfer medium directly. In other examples, use of the organoid medium may be used in combination with the cell differential medium or the organoid transfer medium.

A second embodiment of the instant invention describes a composition for a soft polysaccharide hydrogel capable of conversion to a hard polysaccharide hydrogel and suitable for injection uses. The soft polysaccharide hydrogel comprises one or more water soluble high acyl gellan gum polymers, one or more water soluble low acyl gellan gum polymers, and one or more water soluble chemically modified gellan gum polymers or one or more peptide modified gellan gum polymers. The soft polysaccharide hydrogel exhibits a homogenous matrix structure and the hard polysaccharide hydrogel exhibits an aggregated matrix network structure.

In examples, the soft polysaccharide hydrogel exhibits shear-thinning and self-healing rheological properties, by allowing the soft polysaccharide hydrogel to be converted into a free-flowing (injectable) state by a shearing force, or to recover its hydrogel state once the shearing force is ceased. The shearing force is exerted by pipetting, syringe injecting, and/or pump perfusion.

The soft polysaccharide hydrogel is converted into the hard polysaccharide hydrogel by submersion in an aqueous solution of extra phosphate buffer, submersion in cell culture media, submersion in an ionic solution, and/or contact with bodily fluids (biofluids). The hard polysaccharide hydrogel exhibits 3-D gel structures with rheological properties such that when the hard gel is broken by pipetting or shearing, the hard gel breaks into smaller gel particles, and has an affinity for one or more bioactive molecules and/or cells (e.g., for use in cell therapy). Moreover, there is a potential of cell-biomatrix interaction.

Each bioactive molecule of the one or more bioactive molecules are in contact with, adhered to, suspended in, entrapped in, or embedded in the soft polysaccharide hydrogel and the hard polysaccharide hydrogel while maintaining their bioactivities. Each of the one or more bioactive molecules can release out from or move into the hydrogel. Moreover, the cells in the hydrogel maintain their bioactivities, grow in the hydrogel, and/or differentiate for functional cells or an organoid before or after in vivo injection.

Moreover, the hard polysaccharide hydrogel has a storage modulus value greater than approximately 10 Pa. Further, the hard polysaccharide hydrogel is stiff and brittle. Additionally, the hard polysaccharide hydrogel maintains its gel formation at a temperature equal to or below approximately 80° C., but is capable of being broken into smaller gel particles when disturbed with an external force.

In general, the present invention succeeds in conferring the following benefits and objectives.

It is an object of the present invention to remedy drawbacks to using traditional 3D cell cultures.

It is an object of the present invention to introduce methods using synthetic hydrogels in an organoid environment to produce enhanced control over experimental conditions.

It is an object of the present invention to provide a technology that can be adapted for a liquid handling machine, 3D bioprinting, and/or a microfluid device for lab automation and high-throughput screening, and further, move from an in vitro to an in vivo application as the biocompatible injectable material.

### BRIEF DESCRIPTION OF THE DRAWINGS

FIG. 1 depicts a block diagram of a first method using a hydrogel for an organoid culture, according to at least some embodiments described herein.

FIG. 2 depicts a block diagram of a second method using a hydrogel for an organoid culture, according to at least some embodiments described herein.

FIG. 3 depicts a schematic diagram depicting a method of FIG. 1, according to at least some embodiments described herein.

FIG. 4 depicts a schematic diagram depicting a method of FIG. 2, according to at least some embodiments described herein.

FIG. 5 depicts cell spheroid formation on a first day, a fourth day, and a sixth day, according to at least some embodiments described herein.

FIG. 6A depicts an organoid culture on day zero, a first day, and a third day, according to at least some embodiments described herein.

FIG. 6B depicts an organoid culture on a fifth day and a seventh day, according to at least some embodiments described herein.

FIG. 7A depicts growth of an organoid after adding an organoid medium on a fourth day, a fifth day, and a sixth day, according to at least some embodiments described herein.

FIG. 7B depicts growth of an organoid after adding an organoid medium on a seventh day and an eighth day, according to at least some embodiments described herein.

FIG. 8 depicts organoids grown from cells, according to at least some embodiments described herein.

FIG. 9 depicts images of a live/dead cell viability assay depicting stem cells growing in a hydrogel, according to at least some embodiments described herein.

FIG. 10 depicts images of an immunostaining assay with stem cell biomarkers indicating stem cells growing in a hydrogel, according to at least some embodiments described herein.

FIG. 11 depicts animal injection study images and images from a hematoxylin and eosin or a haematoxylin and eosin (H&E) stain, according to at least some embodiments described herein.

FIG. 12 depicts images of degradation of a 1:0 dilution of hydrogel modified with a matrix metallopeptidase (MMP) enzyme, according to at least some embodiments described herein.

FIG. 13 depicts images of degradation of a 1:3 dilution of hydrogel modified with a matrix metallopeptidase (MMP) enzyme, according to at least some embodiments described herein.

FIG. 14 depicts a graphical representation of a 1:3 dilution of hydrogel modified with a matrix metallopeptidase (MMP) enzyme, according to at least some embodiments described herein.

FIG. 15 depicts a graphical representation of a molecular diffusion test using an azo dye to depict molecular movement in and out of a hydrogel for a 1:0, a 1:1, and a 1:2 dilution, according to at least some embodiments described herein.

FIG. 16 depicts a graphical representation of a molecular diffusion test to depict molecular movement in and out of a hydrogel for a 1:0 dilution, according to at least some embodiments described herein.

FIG. 17 depicts a graphical representation of shear-thinning and rapid recovery rheological properties of hydrogel, according to at least some embodiments described herein.

FIG. 18 depicts a schematic representation of using a xenograft method to culture a cell or induce to an organoid, or harvesting xenograft cells and further culturing or inducing to the organoid, according to at least some embodiments described herein.

### DESCRIPTION OF THE PREFERRED EMBODIMENTS

Gellan gum is a water-soluble anionic capsular polysaccharide produced by the bacterium Sphingomonas elodea (formerly Pseudomonas elodea). The gellan-producing bacterium was discovered and isolated by the former Kelco Division of Merck & Company, Inc. in 1978 from the lily plant tissue from a natural pond in Pennsylvania, USA. It was initially identified as a substitute gelling agent at significantly lower use level to replace agar in solid culture media for the growth of various microorganisms. (Kang K. S., et al., Agar-like polysaccharide produced by a Pseudomonas species: Production and basic properties. Applied & Environmental Microbiology, 1982 43: 1086-1091). The initial gellan gum commercial product with the trademark as “GELRITE” was subsequently identified as a suitable agar substitute as gelling agent in various clinical bacteriological media. (Shungu D, et al., GELRITE as an Agar Substitute in Bacteriological Media, Appl. Environ Microbiol. 1983 46(4): 840-5) As a food additive, gellan gum was first approved for food use in Japan (1988). Subsequently, gellan gum has been approved for food, non-food, cosmetic and pharmaceutical uses by many other countries such as U.S., Canada, China, Korea, European Union, etc. It is widely used as a thickener, emulsifier, and stabilizer.

Gellan gum is manufactured by fermenting an appropriate strain of Sphingomonas with a readily available carbohydrate source. The constituent sugars of gellan gum are glucose, glucuronic acid and rhamnose in the molar ratio of 2:1:1. These are linked together to give a primary structure comprising a linear tetrasaccharide repeat unit (O'Neill M. A. et al., Carbohydrate Research, Vol. 124, p. 123, 1983; and Jansson, P. E. et al., Carbohydrate Research, Vol. 124, p. 135, 1983). In the native or high acyl form of gellan gum, two acyl substituents, acetate and glycerate, are present. Both substituents are located on the same glucose residue and, on average, there is one glycerate per repeat unit and one acetate per every two repeat units. In the low acyl form of gellan gum, the acyl groups have been removed to produce a linear repeat unit substantially lacking such groups. Deacylation of the gum is usually carried out by treating a fermentation broth with alkali.

Shown below in Table 1 are gellan gum (of molecular weights ranging from 5×10⁴ Da to 2×10⁶ Da) with different level of acyl (A-high acyl gellan gum), no/low acyl gellan gum (B) or chemical modified gellan gum such as methacrylated gellan gum (C).

TABLE 1

| | |
|---|---|
| 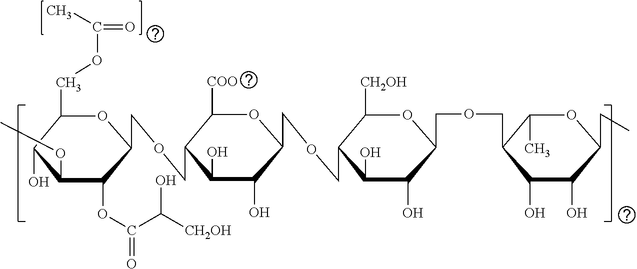 | A |
| 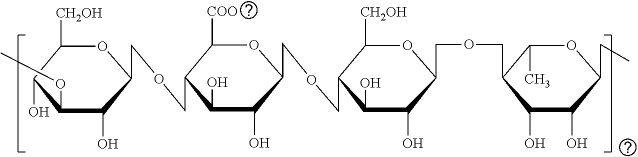 | B |
| 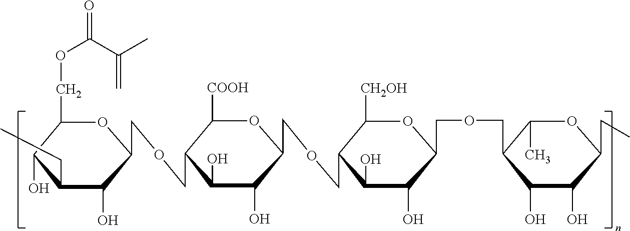 | C |

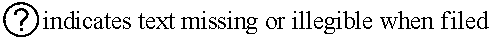

The high acyl form of gellan gum does not require addition of any substances for gel formation provided the gum concentration is higher than the critical concentration. High acyl gellan gum produces a soft, elastic, and non-brittle gel when its solution is cooled below the setting temperature. High acyl gellan gum gels will soften with heat and melt at a temperature proximate to the setting temperature. Low acyl gellan gum polymers typically have a range of the degree of acylation from about 1 to 2 glycerate per repeat and 1 to 2 acetate per every two repeats. The low acyl form of gellan gum generally requires a gelation agent such as salt or acid for gel formation. For example, low acyl gellan gum forms a firm, non-elastic, and brittle gel when cooled in the presence of gel-promoting cations, preferably divalent cations, such as calcium and magnesium.

In general, gellan gum as described above can dissolve in water at the temperature higher than 0° C. at a concentration of 0.001% to 10% w/v, while gellan gum of all types can dissolve completely in water at a temperature higher than 80° C. The gellan gum aqueous solution thus formed can maintain in a liquid form after dissolution or heating-cooling circle at temperature higher than 0° C. and a pH of about 4-10.

The gellan gum as described above can be modified on a position of carboxyl moiety with functional peptides or moleculars through convelant bond. Such modifications can be performed by heating the gellan gum and peptide/molecules mixing solution to 121° C. or higher temperature at high pressure (such as 15 psi) for a time of 3 minutes or longer. An additional method to modify the gellan gum may include using an ethyl(dimethylaminopropyl) carbodiimide (EDC) and N-Hydroxysuccinimide (NETS) or an N-hydroxysulfosuccinimide (Sulfo-NHS) coupling method. In addition, the aforementioned gellan gum solution can also be mixed with functional peptide or moleculars without convelant binding.

The present invention provides a composition for preparing a polysaccharide hydrogel, where one or more chemical molecules modifying the gellan sum are selected from the group consisting of: a) organic molecules that are selected from the group consisting of: polymers of natural or synthetic origin, chemically modified or co-polymers, polypeptide, hyaluronate, chitosan, collagen, polyethyleneglycol anticoagulants, contrasting agents, chemotherapeutic agents, and signaling pathway molecules; and b) inorganic molecules that are selected from the group consisting of: bioactive glass, hydroxyapatite, calcium phosphate and iron.

According to one embodiment of the present invention, the water soluble low acyl gellan gum, high acyl gellan gum, modified gellan gum and a mixture of gellan gum mixture with other chemical/biological molecules, as described above, are suitable for such applications of gellan gum for cell culture and other biomedical application. The selected group of gellan gum can dissolve in water or maintain dissolved in liquid form at room temperature, perform a neutral pH (pH 4-10) and keep the liquid or semi-gel state when surrounding temperature is at or above refrigerator temperature. The gellan gum solution can have various concentrations of 0.001-10% solid contain or chemical modification (e.g. methacrylate) to achieve higher concentration.

Such biological molecules are selected from the group consisting of: cells, peptides, proteins, lipids, polysaccharides, growth factors, growth hormone, antibodies, enzymes, cell receptors, cell ligands, antibiotics, anti-microbial, anti-fungi, antimycotics, albumin, serum, functional peptide molecules with NH₂, COOH and CONH₂ group comprising: RGD, IKVAV, REDV, YIGSRY, poly Lysine. The water-based solvent used in such a preparation method comprises water, phosphate buffer solution (PBS), saline solution, cell culture medium, ionic solution, albumin, serum and xyloglucan.

The present invention provides a composition for preparing a soft polysaccharide hydrogel capable of conversion to a hard polysaccharide hydrogel and suitable for injection uses. The soft polysaccharide hydrogel comprises one or more water soluble high acyl gellan gum polymers, one or more water soluble low acyl gellan gum polymers, and one or more water soluble chemically modified gellan gum polymers or one or more peptide modified gellan gum polymers. The soft polysaccharide hydrogel exhibits a homogenous matrix structure and the hard polysaccharide hydrogel exhibits an aggregated matrix network structure.

In examples, the soft polysaccharide hydrogel exhibits shear-thinning and self-healing rheological properties, by allowing the soft polysaccharide hydrogel to be converted into a free-flowing (injectable) state by a shearing force, or to recover its hydrogel state once the shearing force is ceased. The shearing force is exerted by pipetting, syringe injecting, and/or pump perfusion.

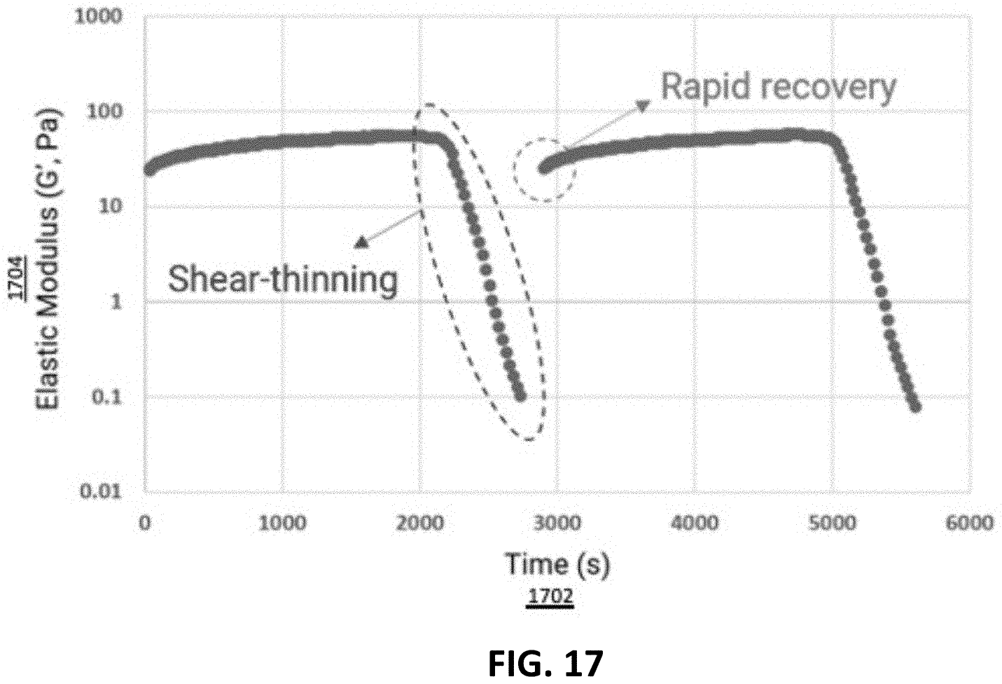

FIG. 17 depicts a graphical representation of the shear-thinning and rapid recovery rheological properties of the hydrogel described herein. As depicted in FIG. 17, the graphical representation includes an x-axis 1702 that measures time in minutes and a y-axis 1704 that measures an elastic modulus in Pa. As depicted, under the mechanical shearing force, such as an injection through a syringe, the hydrogel performs a gel-sol transition and becomes a free-flowing and injectable state. However, once the shearing force ceases, the mechanical strength of the hydrogel rapidly recovers with a sol-gel transition and becomes the hydrogel again. With this injectable property, the hydrogel can be used for in vivo cells/drug delivery with great retention, bioprinting, droplet formation, and lab automation.

The soft polysaccharide hydrogel is converted into the hard polysaccharide hydrogel by submersion in an aqueous solution of extra phosphate buffer, submersion in cell culture media, submersion in an ionic solution, and/or contact with bodily fluids (biofluids). The hard polysaccharide hydrogel exhibits 3-D gel structures with rheological properties such that when the hard gel is broken by pipetting or shearing, the hard gel breaks into smaller gel particles, and has an affinity for one or more bioactive molecules or cells. Each bioactive molecule of the one or more bioactive molecules are in contact with, adhered to, suspended in, entrapped in, or embedded in the soft polysaccharide hydrogel and the hard polysaccharide hydrogel while maintaining their bioactivities.

Moreover, the hard polysaccharide hydrogel has a storage modulus value greater than approximately 10 Pa. Further, the hard polysaccharide hydrogel is stiff and brittle. Additionally, the hard polysaccharide hydrogel maintains its gel formation at a temperature equal to or below approximately 80° C., but is capable of being broken into smaller gel particles when disturbed with an external force.

In some examples, the composition of the soft polysaccharide hydrogel comprises from about 0.001% to about 20% of the one or more high acyl gellan gum polymers, about 0.001% to about 20% of the one or more low acyl gellan gum polymers, about 0.001% to about 20% of the one or more modified gellan gum polymers, and further comprises from about 0.00001% to about 30% of the one or more bioactive molecules. In a preferred embodiment, the composition comprises from about 0.01% to about 5% of the one or more high acyl gellan gum polymers, about 0.01% to about 5% of the one or more low acyl gellan gum polymers, about 0.01% to about 5% of the one or more modified gellan gum polymers, and further comprises from about 0.001% to about 10% of the one or more bioactive molecules.

The gellan gum solution can be trigger into hydrogel by directly mixing with water-based solvents, which include, but are not limited to, phosphate buffer (PBS), cell culture media or ionic solutions at a temperature ranging from about 4° C. to about 60° C. The storage modulus (G′) of the system increases upon mixing and surpasses about 10 Pa within 30 min, and in a preferred embodiment, storage modulus (G′) of the system surpasses about 10 to 20000 Pa, which indicate the system is stronger enough to suspend cells within its hydrogel matrix for 3D growth. The trigger solution using for this hydrogel formation can be any type of cell culture media with or without serum, buffers, ionic solutions with pure or mixture of mono, divalent or polyvalent cations or the mixture of above solutions. Overall, the hydrogel described herein can be used for 3D cell cultures, 2D coatings, carriers for different bioactive molecules for slow release, injection, bioprinting, etc.

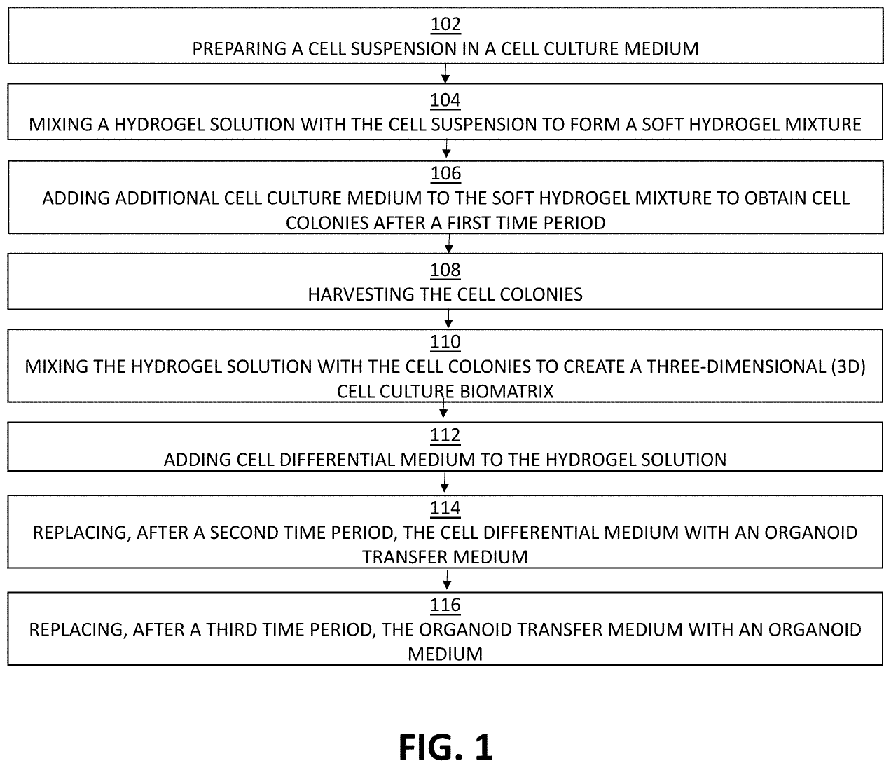

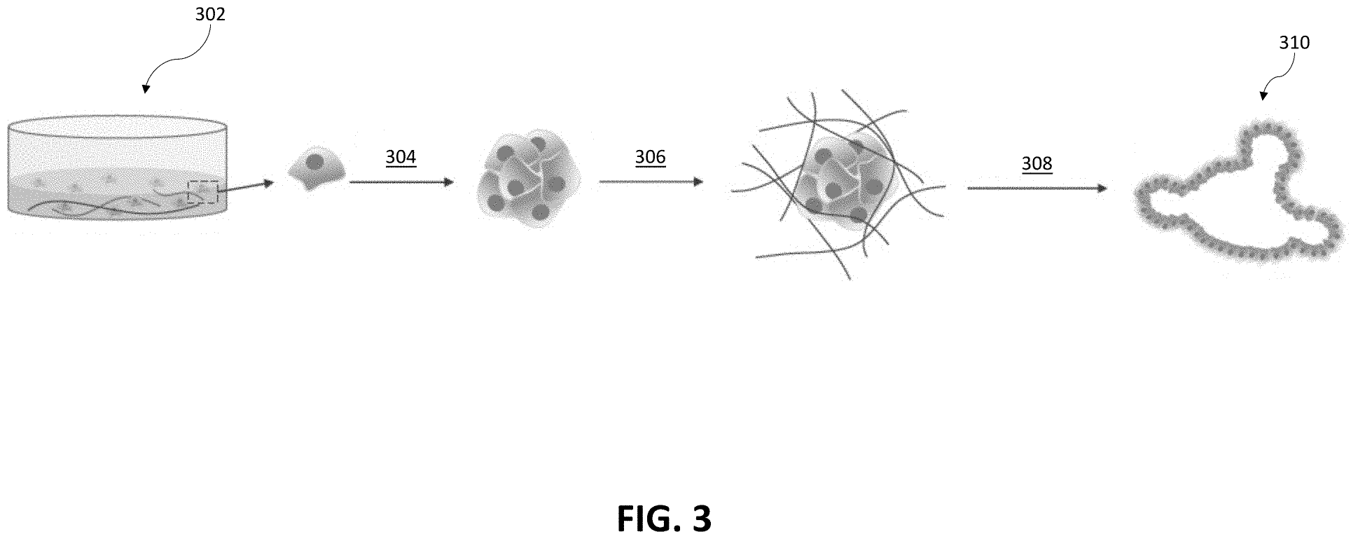

A first method using the soft polysaccharide hydrogel described above for an organoid culture is depicted in FIG. 1. The first method of FIG. 1 is depicted schematically in FIG. 3. The method of FIG. 1 begins at a process step 102 that includes preparing a cell suspension in a cell culture medium. The cell suspension comprises single stem cells, stem cell clusters, cells from an isolated tissue, cells from an organoid fragment, cells from a cell line, cells from blood, and/or cells from a xenograft sample, among other cells not explicitly listed herein.

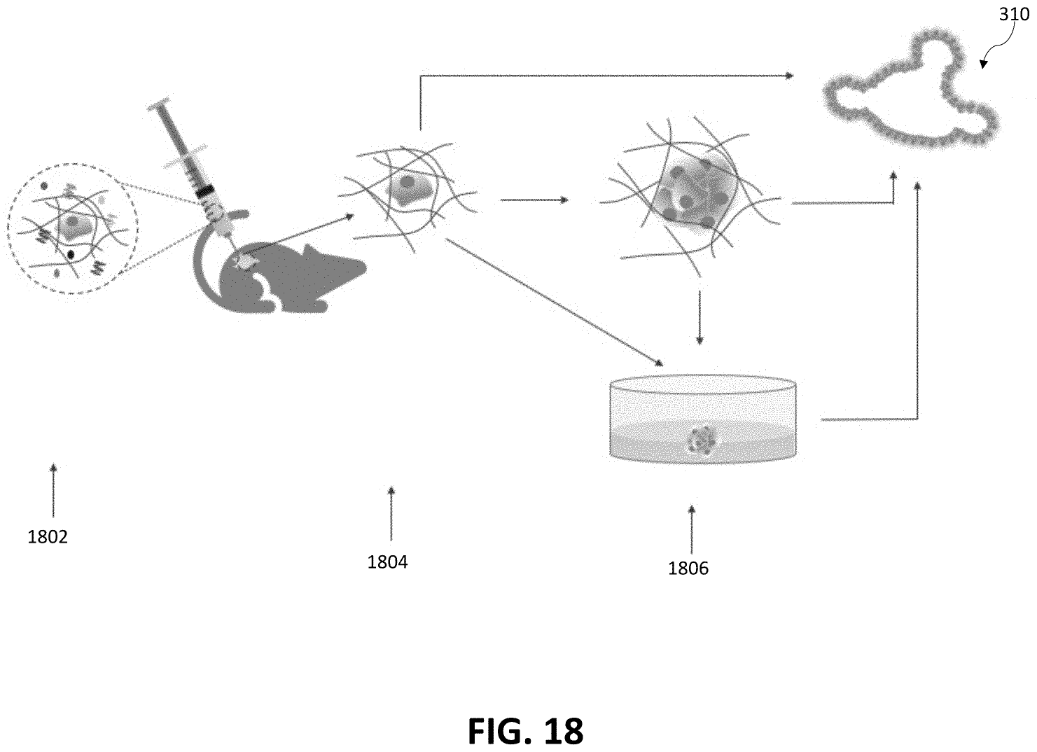

Regarding the cells from the xenograft culture method, and as depicted in FIG. 18, cells may first be isolated from patient tissue and may be suspended in a hydrogel (a process step 1802). In the process step 1802, growth factors and/or small molecules, such as the ones described herein, may also be mixed into the hydrogel. A process step 1804 follows the process step 1802, where the cells may be injected to an animal (xenograft). The injected cells maintain their bioactivities, grow in the hydrogel, or differentiate for functional cells or the organoid 310 before or after in vivo injection. A process step 1806 follows the process step 1804, where the xenograft cells may be harvested out from the animal to achieve a higher cell number or more mature cells or to induce to the organoid 310.

The process step 102 of the method of FIG. 1 is followed by a process step 104 that includes mixing a hydrogel solution with the cell suspension (of the process step 102) to form a soft hydrogel mixture. It should be appreciated that the cells in the hydrogel maintain their bioactivities, grow in the hydrogel, or differentiate for functional cells or the organoid 310 before or after in vivo injection.

An elastic modulus of the hydrogel solution is in a range between approximately 0.001 Pa to approximately 5000 Pa. In some examples, the elastic modulus of the soft hydrogel mixture is in a range between approximately 0.01 Pa to approximately 2000 Pa. In other examples, the elastic modulus of the soft hydrogel mixture is less than 50 Pa when the cell colony needs to be formed quickly and then the organoid inducement is started from the cell colony. In other examples, the cells may be encapsulated in the hydrogel biomatrix (having an elastic modulus between 1 Pa to 5000 Pa) and then a medium is added to the hydrogel biomatrix to induce the organoid directly.

In some examples, an inhibitor/small molecule and/or a growth factor may be added to the soft hydrogel mixture. The inhibitor/small molecule may include ROCK1, Thiazovicin, CHIR99021, LY294002, A 83-01, Nicotinamide, SB 202190, Gastrin, DAPT, Forskolin, Prostaglandin E2, Testisteribe, SB 431542, retinoic acid, Y27632, MD2206, Dorsomorphin, G27632, and/or the smoothened agonist, among others not explicitly listed herein. The growth factor may include: the EGF, the IGF, the FGF, R-Spondin, Wnt-3a, the BMP, the hepatocyte growth factor, Activin A, the dickkopf-related protein, the brain-derived neurotrophic factor, the glial cell-derived neurotrophic factor, the sonic hedgehog, heregullin, prolactin, and/or Noggin, among others not explicitly listed herein. It should be appreciated that in some examples, the inhibitor/small molecule and/or the growth factor may be added to the hydrogel prior to injection.

In some examples, the process step 104 of FIG. 1 may be followed by the following process step: modifying the hydrogel solution with a functional ligand and/or a functional peptide. The functional ligand may include: RGD, a matrix metallopeptidase (MMP) sensitive ligand, a laminin functional ligand, a vitronectin functional ligand, a fibronectin functional ligand, an osteopontin functional ligand, a nidogen functional ligand, an elastin functional ligand, a thrombospondin functional ligand, and/or a collagen functional ligand, among others not explicitly listed herein. The laminin functional ligand may be YIGSR and/or IKAVA.

The functional peptide may include a MMP functional peptide, a collagen functional peptide, a vitronectin functional peptide, laminin functional peptide, and/or a functional peptide molecule having an amine group, a carboxyl group, and an amide group, among others not explicitly listed herein. The functional peptide molecule having an amine group, the carboxyl group, and the amide group may include RGD, IKVAV, REDV, YIGSRY, and/or poly Lysin, among others not explicitly listed herein. It should be appreciated that the hydrogel solution may be modified with other functional ligands and/or functional peptides that are not described herein.

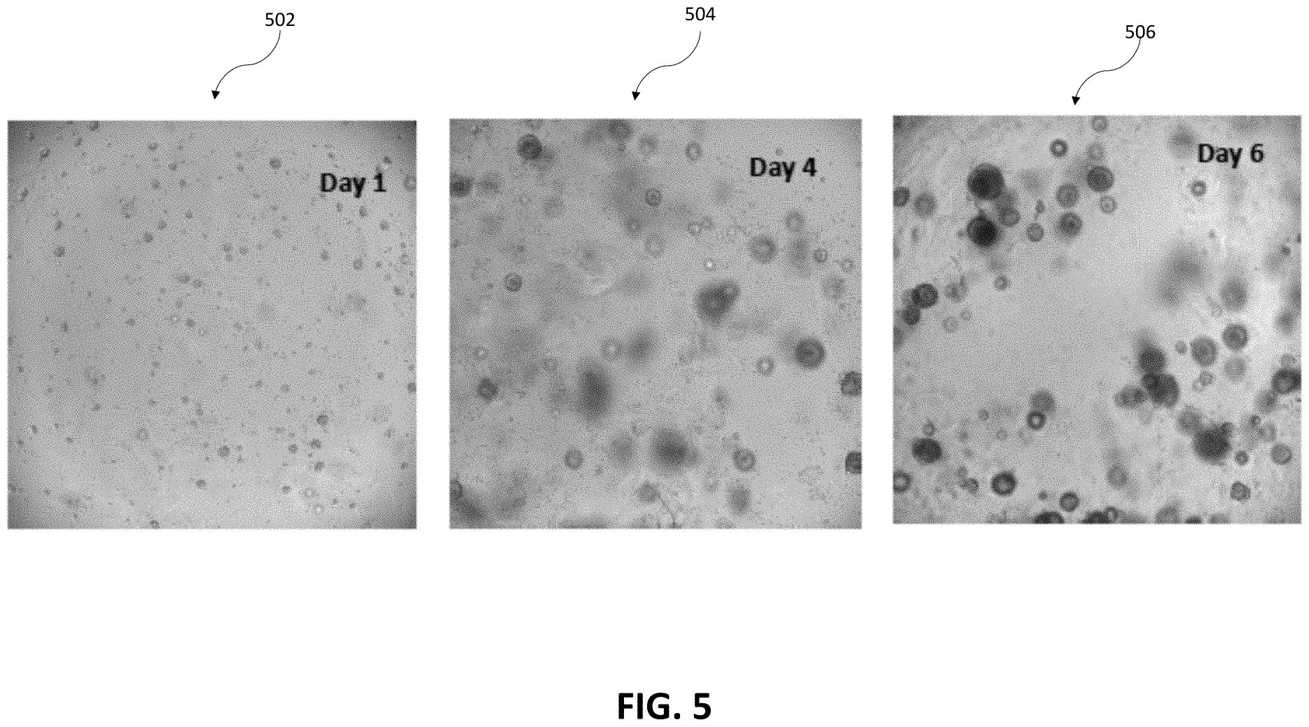

As depicted in FIG. 3, the soft polysaccharide hydrogel may be used for a quick spheroid formation at a process step 302. As described herein, “spheroids” are a type of 3D cell modeling that better simulate a live cell's environmental conditions compared to a 2D cell model, specifically with the reactions between cells and the reactions between cells and the matrix. As depicted in FIG. 3, cell spheroid formation may occur (at a process step 304). The cell spheroid formation may also be depicted in FIG. 5 at a first day 502, a fourth day 504, and a sixth day 506.

The process step 104 of FIG. 1 is followed by a process step 106 that includes adding the soft hydrogel mixture of the process step 104 to a cell culture plate and then adding additional cell culture medium on top of the soft hydrogel mixture to obtain cell colonies after a first time period in a range of approximately one day to approximately seven days for growth of the cell colonies to become a size in a range of approximately 10 micrometers to approximately 1000 micrometers in diameter. The cell colonies may form within the soft hydrogel mixture or at the bottom of the cell culture plate. The cells may become encapsulated in the soft hydrogel mixture (depicted as a process step 306 in FIG. 3).

Next, the process step 106 of FIG. 1 is followed by a process step 108 that includes harvesting the cell colonies and mixing the hydrogel solution with the cell colonies to create a 3D cell culture biomatrix. As described herein a “biomatrix” is a biological or biochemical matrix, or set of interacting systems. The elastic modulus of the 3D cell culture biomatrix is in the range between approximately 1 Pa to approximately 5000 Pa. The cells of the cell suspension embedded or cultured in the 3D cell culture biomatrix are injectable for in vivo application. Injected stem cells, cells from patient-derived tissue, organoid cells, etc. can survive, grow, differentiate, or become the organoid after injection.

The process step 108 may be followed by a process step 110 that includes adding inhibitors/small molecules and/or growth factor proteins to the cell culture biomatrix. The inhibitor/small molecule may include ROCK1, Thiazovicin, CHIR99021, LY294002, A 83-01, Nicotinamide, SB 202190, Gastrin, DAPT, Forskolin, Prostaglandin E2, Testisteribe, SB 431542, Retinoic acid, Y27632, MD2206, Dorsomorphin, G27632, and/or a smoothened agonist, among others not explicitly listed herein. The growth factor may include: an EGF, an IGF, a FGF, R-Spondin, Wnt-3a, a BMP, a hepatocyte growth factor, Activin A, a dickkopf-related protein, a brain-derived neurotrophic factor, a glial cell-derived neurotrophic factor, sonic hedgehog, heregullin, prolactin, and/or Noggin, among others. It should be appreciated that the cells of the cell suspension embedded or cultured in the 3D cell culture biomatrix are injectable for in vivo application.

The process step 110 of FIG. 1 may be followed by a process step 112 that includes adding cell differential medium to the hydrogel solution. The cell differential medium comprises the activin A, the FGF, and/or the IGF, among other materials. Addition of the cell differential medium is also depicted as a process step 308 in FIG. 3.

After a second time period in a range of approximately one day to approximately seven days, the process step 112 of FIG. 1 is followed by a process step 114 that includes replacing the cell differential medium with an organoid transfer medium. The organoid transfer medium comprises the activin A, the FGF, Noggin, the EGF, and/or R-spondin-1, among other materials. Moreover, after a third time period in a range of approximately twenty-four hours to approximately forty-eight hours, the process step 114 is followed by a process step 116 of FIG. 1 that includes replacing the organoid transfer medium with an organoid medium. The organoid medium comprises Noggin, the EGF, and/or R-spondin-1, among other materials. Addition of the organoid medium is also depicted as the process step 308 in FIG. 3.

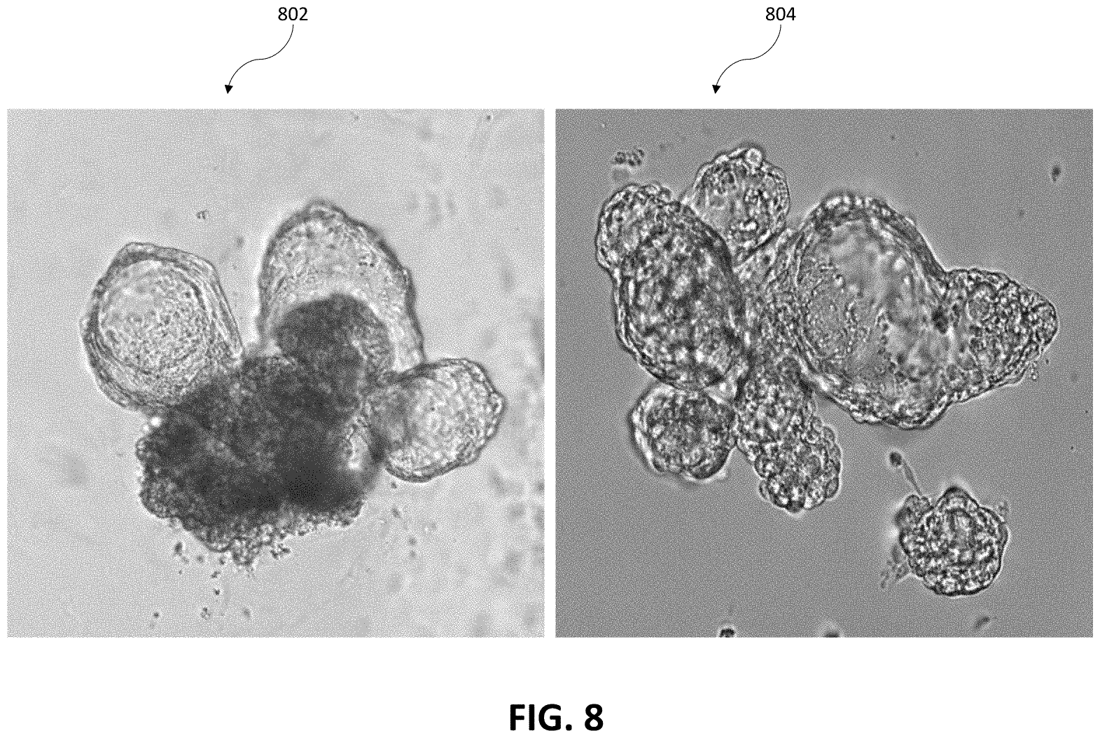

Subsequent the process step 116 of FIG. 1 and the process step 308 of FIG. 3, an organoid 310 of FIG. 3 may be formed. The organoid 310 of FIG. 3 may be depicted as a first image 802 and a second image 804 in FIG. 8. As described herein, an “organoid” is a miniaturized and simplified version of an organ produced in vitro in 3D that shows realistic micro-anatomy. Further, the organoid is a 3D cell culture that incorporates some of the key features of the represented organ. These in vitro culture systems contain a self-renewing stem cell population, which differentiates into multiple, organ-specific cell types that exhibit spatial organization similar to the corresponding organ and are capable of recapitulating some functions of that organ providing a highly physiologically relevant system.

In examples, a universal medium may be used to induce the cells directly to the organoid. In examples, the medium may be reformulated to directly induce single cells or cell colonies to organoid. It should be appreciated that in some examples, the process step 112 of FIG. 1 may be omitted, such that the process step 110 is followed by a variation of the process step 114, which includes adding an organoid transfer medium to the hydrogel solution.

In additional examples, use of the organoid medium may be used in combination with the cell differential medium or the organoid transfer medium. In a first example, a slight variation to the process step 112 may include adding the cell differential medium and the organoid medium to the hydrogel solution. The slight variation to the process step 112 may end the method of FIG. 1. In a second example, a slight variation to the process step 114 may include replacing the cell differential medium with both the organoid transfer medium and the organoid medium. The slight variation to the process step 114 may end the method of FIG. 1.

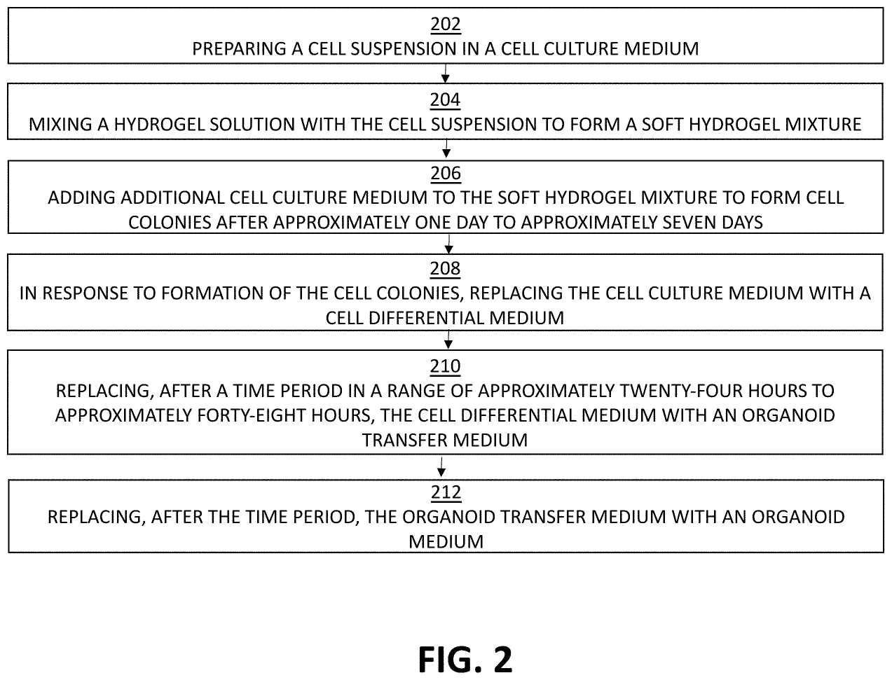

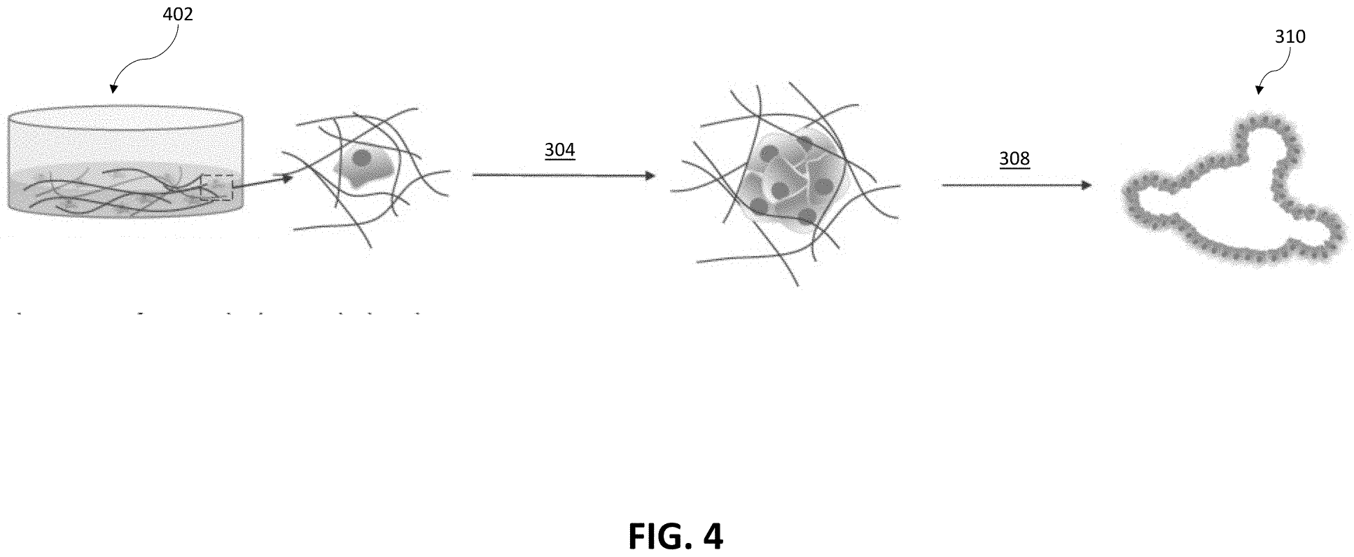

A second method using the soft polysaccharide hydrogel described above for the organoid culture is depicted in FIG. 2. The method of FIG. 2 may be illustratively depicted in FIG. 4. The method of FIG. 2 begins at a process step 202 that includes preparing a cell suspension in a cell culture medium. The cell suspension comprises the single stem cells, the stem cell clusters, the cells from the isolated tissue, the cells from the organoid fragment, the cells from the cell line, the cells from the xenograft sample, and/or the cells from the blood, among other cells not explicitly listed herein.

The process step 202 is followed by a process step 204 of FIG. 2 that includes mixing a hydrogel solution with the cell suspension to form a soft hydrogel mixture. In this example, the cells may be encapsulated directly in the soft hydrogel mixture (depicted as a process step 402 of FIG. 4). In some examples, the cells may be embedded in the hydrogel. As depicted in FIG. 4, cell spheroid formation may occur (at the process step 304). The cell spheroid formation may also be depicted in FIG. 5 at a first day 502, a fourth day 504, and a sixth day 506.

In some examples, the process step 204 of FIG. 2 may be followed by a process step (not shown) that includes modifying the hydrogel solution with a functional ligand and/or a functional peptide, as described above. It should be appreciated that the hydrogel solution may be modified with other functional ligands and/or functional peptides that are not described herein.

The process step 204 of FIG. 2 may be followed by a process step 206 that includes adding the soft hydrogel mixture to a cell culture plate and adding additional cell culture medium to the soft hydrogel mixture to form cell colonies after approximately one day to approximately seven days. In response to formation of the cell colonies, the process step 206 of FIG. 2 may be followed by a process step 208 that includes replacing the cell culture medium with a cell differential medium. The cell differential medium may include: activin A, the FGF, and/or the IGF, among other materials not explicitly listed herein. The addition of the cell differential medium may also be depicted in the process step 308 of FIG. 4.

After a time period in a range of approximately twenty-four hours to approximately forty-eight hours, the process step 208 may be followed by a process step 210 of FIG. 2 that includes replacing the cell differential medium with an organoid transfer medium. The organoid transfer medium comprises activin A, the FGF, Noggin, the EGF, retinoic acid, and/or R-spondin-1, among other materials not explicitly listed herein. Next, after the time period in the range of approximately twenty-four hours to approximately forty-eight hours, the process step 210 of FIG. 2 may be replaced with a process step 212 that includes replacing the organoid transfer medium with an organoid medium. The organoid medium comprises Noggin, the EGF, and/or R-spondin-1, among other materials not explicitly listed herein. The addition of the organoid medium may also be depicted in the process step 308 of FIG. 4.

Subsequent the process step 212 of FIG. 2 and the process step 308 of FIG. 4, the organoid 310 of FIG. 3 may be formed. The organoid 310 of FIG. 3 may be depicted as the first image 802 and the second image 804 in FIG. 8.

In examples, a universal medium may be used to induce the cells directly to the organoid. In examples, the medium may be reformulated to directly induce single cells or cell colonies to organoid. It should be appreciated that in some examples, use of the cell differential medium is not needed (e.g., the process step 208 of FIG. 2) and one may use of the organoid transfer medium directly. In a further example, the universal medium may be used from the process step 206 of FIG. 2.

In other examples, use of the organoid medium may be used in combination with the cell differential medium or the organoid transfer medium. In another example, a variation to the process step 208 of FIG. 2 may be used and may include replacing the cell culture medium with a cell differential medium and the organoid medium in response to formation of the cell colonies. The slight variation to the process step 208 may end the method of FIG. 2. In a further example, a slight variation to the process step 210 may include replacing the cell differential medium with the organoid transfer medium and the organoid medium after the time period in the range of approximately twenty-four hours to approximately forty-eight hours. The slight variation to the process step 210 may end the method of FIG. 2.

It should be appreciated that in some examples, the cell culture medium of FIG. 1 and/or FIG. 2 includes a protein and/or a metabolite of a vitamin A₁ to induce the cells to the organoid. The protein may include activin A, the FGF, and/or the IGF, among others. The metabolite of the vitamin A₁ is R-spondin-1. In other examples, the cell culture medium includes the growth factor/protein and the inhibitor/small molecule to induce the cells to the organoid culture.

The methods of FIG. 1 and FIG. 2 provide a technology that can be adapted for a liquid handling machine, 3D bioprinting, and/or a microfluid device for lab automation and high-throughput screening. Moreover, the methods of FIG. 1 and FIG. 2 provide a technology that moves from an in vitro application to an in vivo application. Furthermore, it should be appreciated that the injected stem cells, the cells from patient-derived tissue, the organoid cells, etc. can survive, grow, differentiate, or become the organoid after injection.

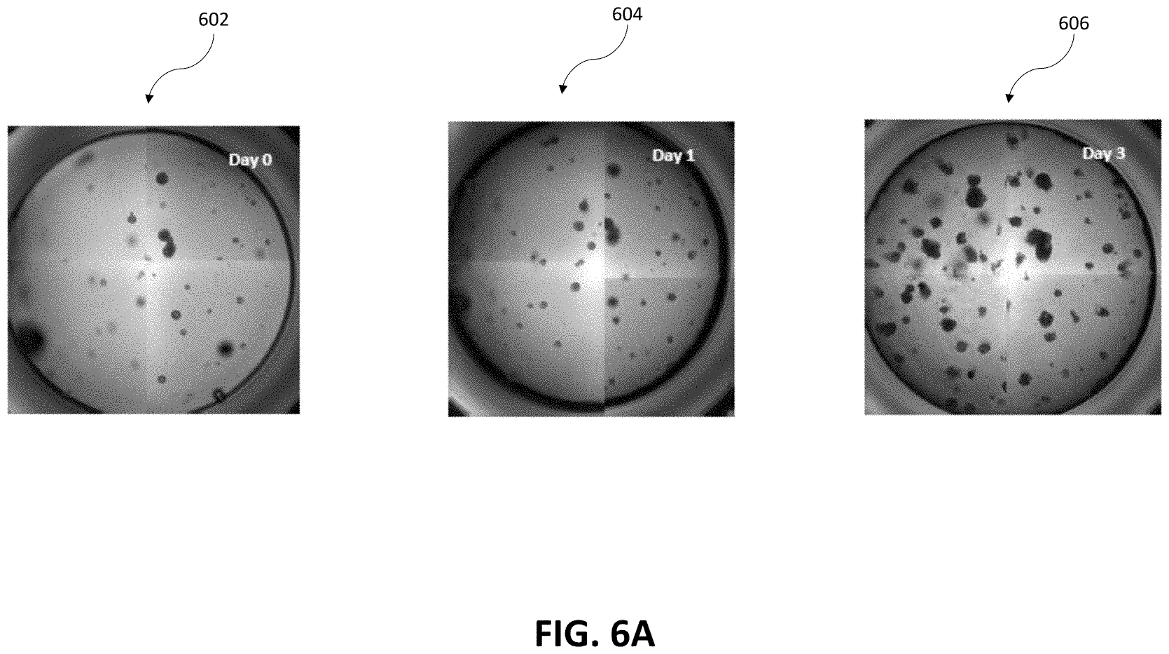

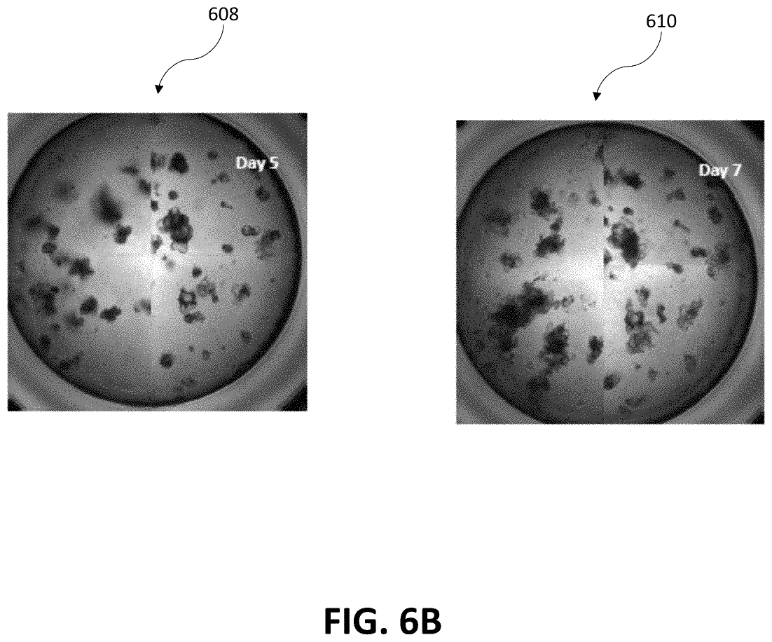

FIG. 6A and FIG. 6B depict the organoid culture 310 formed by the methods of FIG. 1 and FIG. 2. Specifically, FIG. 6A depicts a day zero 602, a first day 604, and a third day 606 of the organoid culture 310. As described herein, the “day zero” describes the day when the encapsulation of the cells or cell colonies in the biomatrix begins for the organoid formation. The day zero 602 depicts encapsulated cell spheroids in the hydrogel. The first day 604 depicts addition of the cell differential medium. Addition of the cell differential medium may also be depicted as the process step 112 of FIG. 1, the process step 208 of FIG. 2, and the process step 308 of FIG. 3 and FIG. 4. The third day 606 depicts the addition of the organoid transfer medium. Addition of the organoid transfer medium may also be depicted as the process step 114 of FIG. 1 and the process step 210 of FIG. 2.

FIG. 6B depicts a fifth day 608 and a seventh day 610. Both the fifth day 608 and the seventh day 610 depict the organoid culture 310 after addition of the organoid medium and continuation of the organoid culture 310 such that the organoid culture 310 can mature. Addition of the organoid medium may also be depicted as the process step 116 of FIG. 1, the process step 212 of FIG. 2, and the process step 308 of FIG. 3 and FIG. 4.

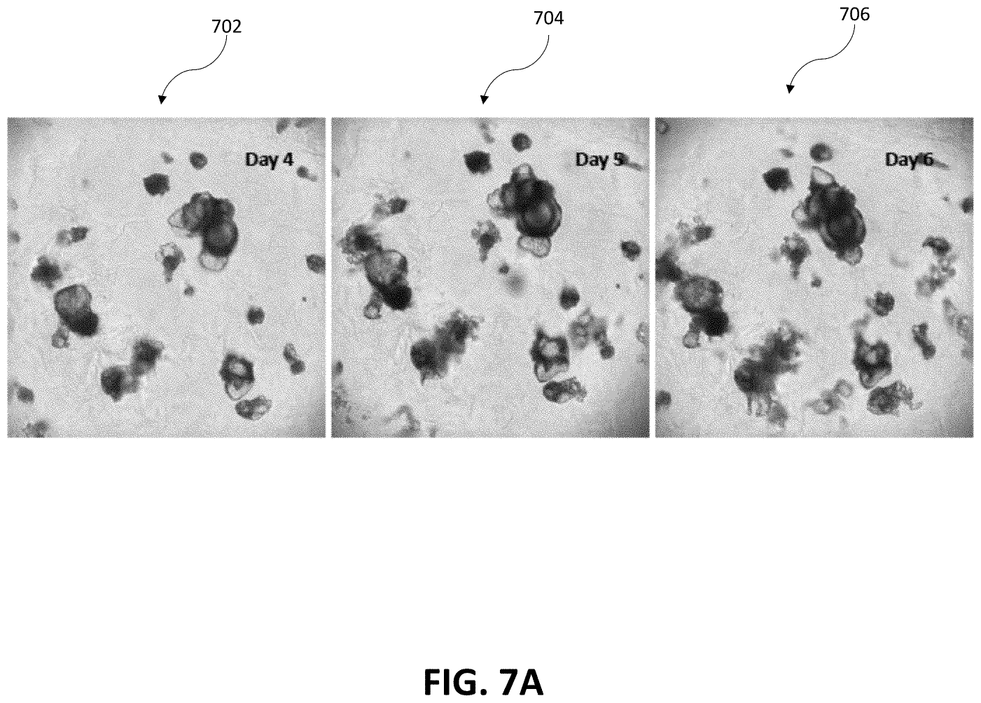

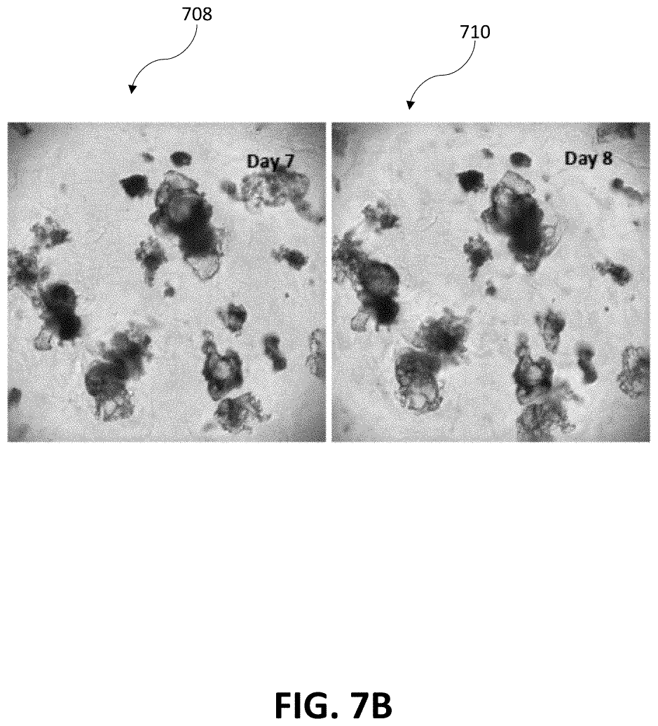

FIG. 7A depicts growth of the organoid culture 310 after the addition of the organoid medium after a fourth day 702, a fifth day 704, and a sixth day 706. FIG. 7B depicts growth of the organoid culture 310 after the addition of the organoid medium after a seventh day 708 and an eighth day 710. Addition of the organoid medium may also be depicted as the process step 116 of FIG. 1, the process step 212 of FIG. 2, and the process step 308 of FIG. 3 and FIG. 4.

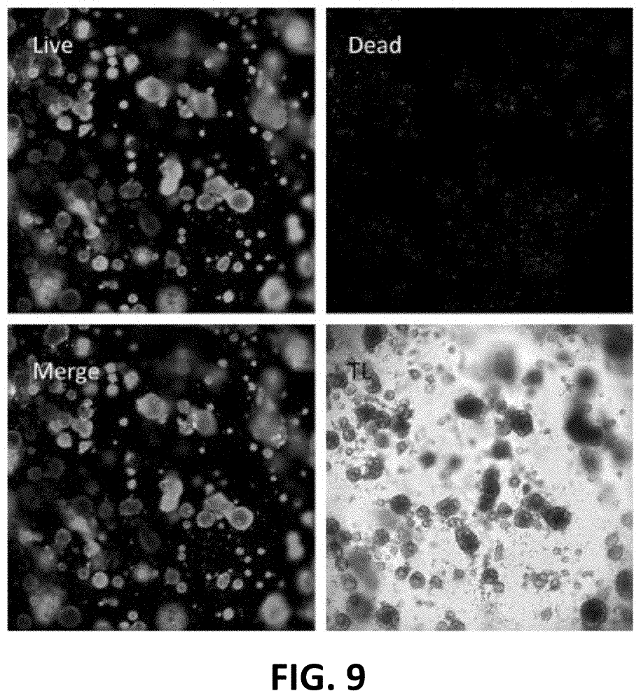

FIG. 9 depicts images of a live/dead cell viability assay showing the stem cells growing in the hydrogel (of the process step 106 of FIG. 1 and the process step 206 of FIG. 2). As described herein, the “live/dead cell viability assay” is a two-color assay used to determine viability of cells in a population based on plasma membrane integrity and esterase activity. The assay may include two fluorescent dyes that differentially label live and dead cells. As such, the assay may be used for the rapid quantitation of cell viability using flow cytometry or fluorescent microscopy. As depicted in FIG. 9, the stem cells growing in the hydrogel maintain a high level of cell viability.

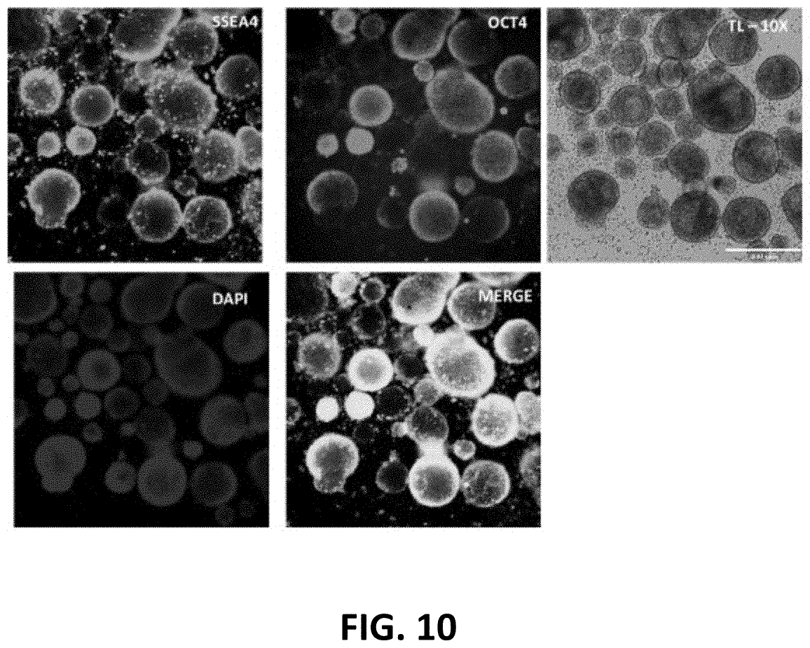

FIG. 10 depicts images of an immunostaining assay with stem cell biomarkers indicating the stem cells growing in the hydrogel (of the process step 106 of FIG. 1 and the process step 206 of FIG. 2). As described herein, “immunostaining” is any use of an antibody-based method to detect a specific protein in a sample. As depicted in the images of FIG. 10, the stem cells growing in the hydrogel maintain a high level of cell differentiation capability.

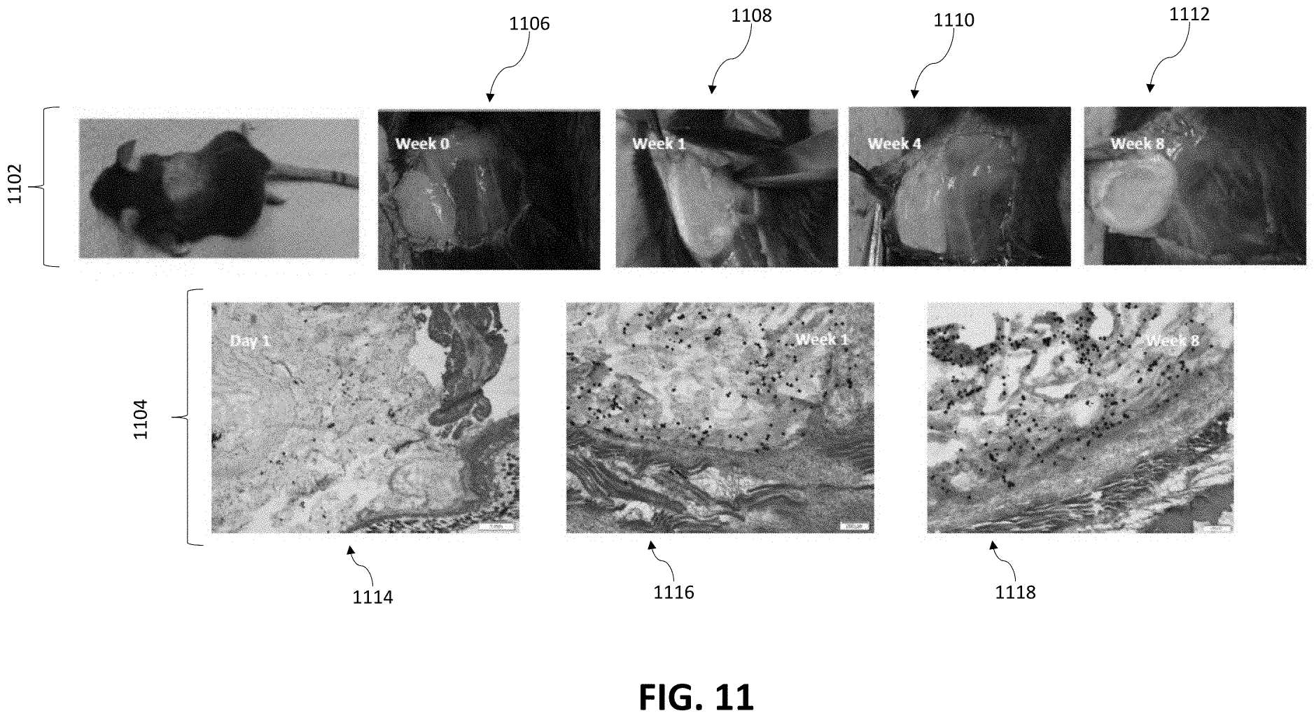

It should be appreciated that the hydrogel used in the method of FIG. 1 and the method of FIG. 2 is biocompatible. FIG. 11 depicts that the hydrogel is stable after injection in an animal 1102. The stability of the hydrogel after injection in the animal 1102 is depicted a week zero 1106, a first week 1108, a fourth week 1110, and an eighth week 1112.

Moreover, FIG. 11 depicts images from a hematoxylin and eosin or a haematoxylin and eosin (H&E) stain 1104. As described herein, the “H&E stain” is a combination of two histological stains: hematoxylin and eosin. The hematoxylin stains cell nuclei blue, and eosin stains the extracellular matrix and cytoplasm pink, with other structures taking on different shades, hues, and combinations of these colors. The stain shows the general layout and distribution of cells and provides a general overview of a tissue sample's structure. As depicted in FIG. 11, the images of the H&E stain 1104 are depicted on a first day 1114, a first week 1116, and an eighth week 1118. The images of the H&E stain 1104 depict health tissue surrounding the hydrogel.

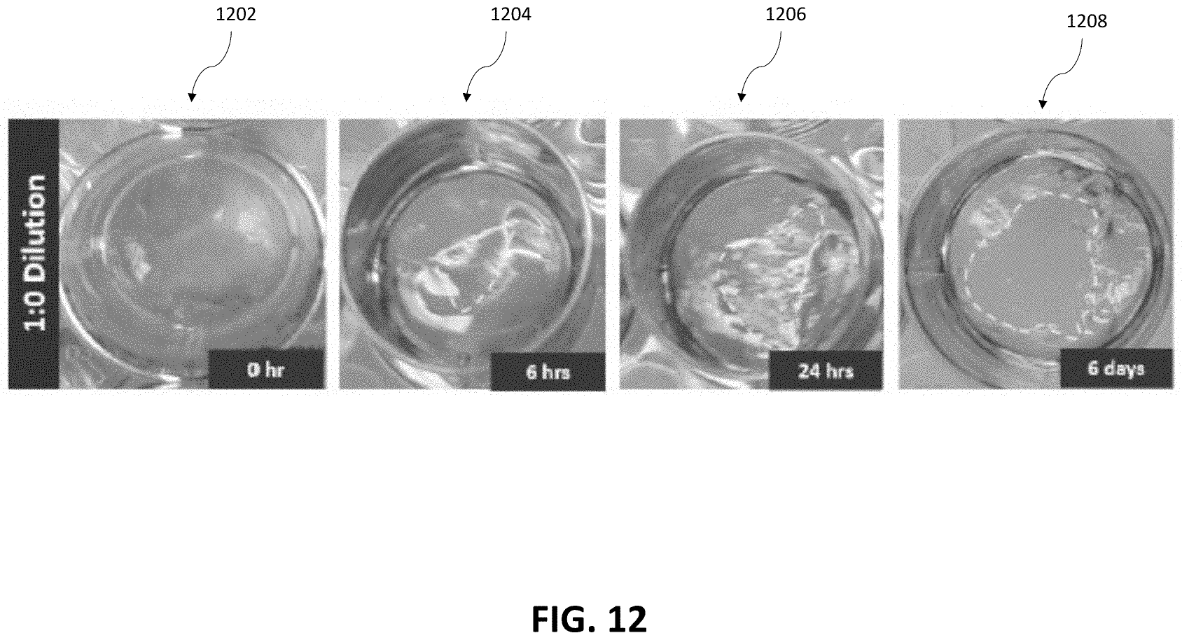

FIG. 12 depicts images of degradation of a 1:0 dilution of hydrogel modified with a MMP enzyme. As described herein, the “MMP” a metalloproteinase that is a calcium-dependent zinc-containing endopeptidase. An MMP enzyme is capable of degrading extracellular matrix proteins, but also can process a number of bioactive molecules. MMPs are involved in the cleavage of cell surface receptors, the release of apoptotic ligands (such as the FAS ligand), and chemokine/cytokine inactivation. MMPs are also thought to play a major role in cell behaviors, such as cell proliferation, migration (adhesion/dispersion), differentiation, angiogenesis, apoptosis, and host defense. MMPs have also been proposed as markers of many pathological conditions for their ability to degrade extracellular matrix components and remodel tissues.

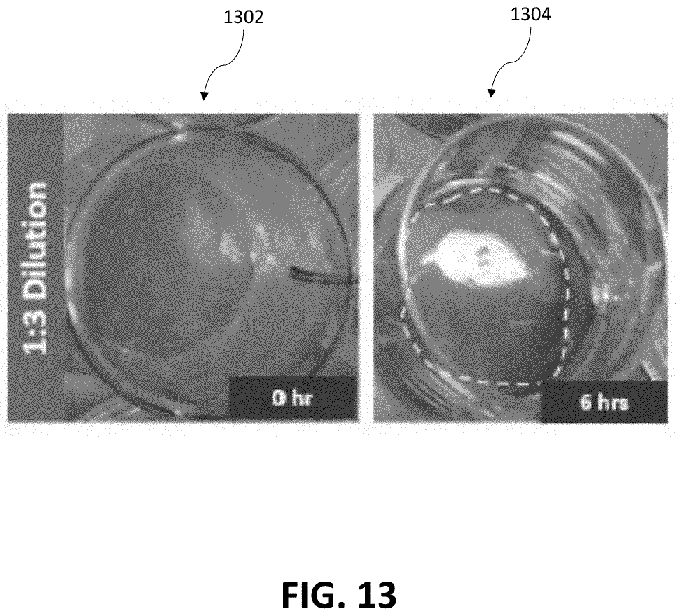

FIG. 12 depicts the images of degradation of the 1:0 dilution of hydrogel modified with the MMP enzyme at a zero hour 1202, after six hours 1204, after twenty-four hours 1206, and after six days 1208. FIG. 13 depicts images of degradation of a 1:3 dilution of hydrogel modified with the MMP enzyme at a zero hour 1302 and after six hours 1304. As depicted in FIG. 12 and FIG. 13, when the hydrogel is modified with the MMP enzyme, the hydrogel becomes enzyme sensitive. The hydrogel also has degradation in the presence of the MMP enzyme. As depicted, the degradation rate may be manipulated by the concentration of the hydrogel and the MMP enzyme.

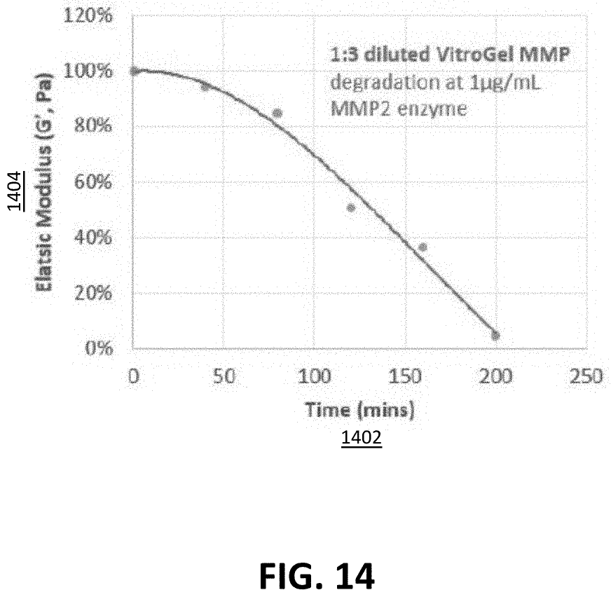

FIG. 14 depicts a graphical representation of a 1:3 dilution of hydrogel modified with the MMP enzyme and degradation at 1 μg/mL. The graph includes an x-axis 1402 and a y-axis 1404. The x-axis 1402 illustrates a time measured in minutes and the y-axis 1404 illustrates an elastic modulus measured in Pa. As depicted, the degradation rate may be manipulated by the concentration of the hydrogel and the MMP enzyme.

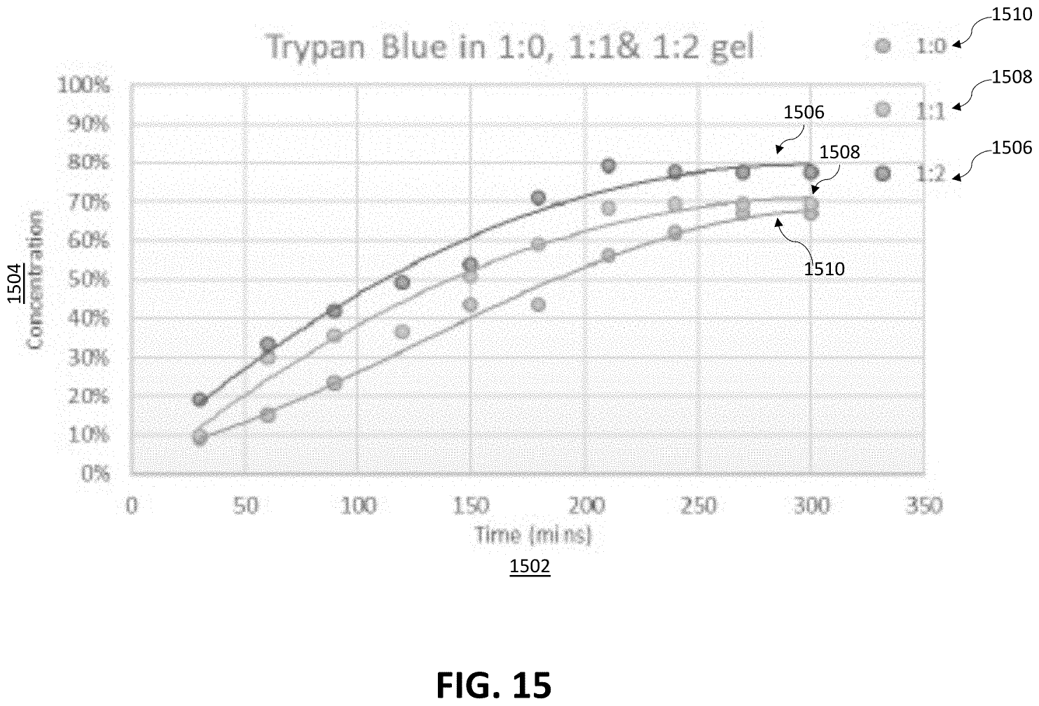

FIG. 15 depicts a graphical representation of a molecular diffusion test using a dye (e.g., trypan blue) to depict molecular movement in and out of the hydrogel for a 1:0, a 1:1, and a 1:2 dilution. It should be appreciated that all types of the hydrogel described herein may be used with this model. In some examples, the hydrogel may be modified with the MMP enzyme. The graph of FIG. 15 includes an x-axis 1502 and a y-axis 1504. The x-axis 1502 depicts time measured in minutes and the y-axis 1504 depicts a concentration measured as a percentage. The graph of FIG. 15 measures the 1:0 dilution 1510 of the hydrogel modified with the MMP enzyme, the 1:1 dilution 1508 of the hydrogel modified with the MMP enzyme, and the 1:2 dilution 1506 of the hydrogel modified with the MMP enzyme. Thus, as depicted, the mobility of the molecule/protein is dependent on the hydrogel concentration, as well as the size of the molecules.

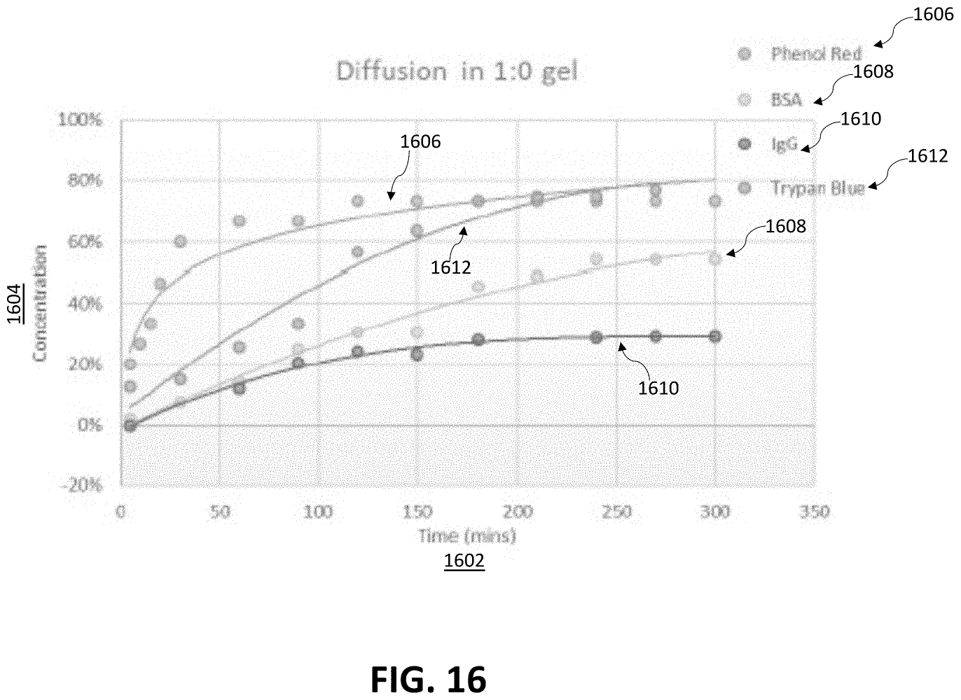

FIG. 16 depicts another graphical representation of the molecular diffusion test to depict molecular movement in and out of the hydrogel for a 1:0 dilution. It should be appreciated that all types of the hydrogel described herein may be used with this model. In some examples, the hydrogel may be modified with the MMP enzyme. The graph of FIG. 16 includes an x-axis 1602 and a y-axis 1604. The x-axis 1602 depicts time measured in minutes and the y-axis 1604 depicts a concentration measured as a percentage. The graph of FIG. 16 measures the 1:0 dilution of the hydrogel modified with the MMP enzyme using a pH indicator (phenol red 1606), an organosilicon compound with the formula Me₃SiNC(OSiMe₃)Me (Me=CH₃) (bis(trimethylsilyl)acetamide (BSA) 1608), an antibody (Immunoglobulin G (IgG) 1610), and an organosulfonate salt that is the tetrasodium salt of 3,3′-[(3,3′-dimethylbiphenyl-4,4′-diyl)didiazene-2,1-diyl]bis(5-amino-4-hydroxynaphthalene-2,7-disulfonic acid) (trypan blue 1612). Thus, as depicted, the mobility of the molecule/protein is dependent on the hydrogel concentration, as well as the size of the molecules.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method using a soft polysaccharide hydrogel for an organoid culture, the method comprising:
preparing a cell suspension in a cell culture medium, wherein the cell suspension comprises single stem cells, stem cell clusters, cells from an isolated tissue, cells from an organoid fragment, cells from a cell line, cells from a xenograft sample, and/or cells from blood;
mixing a hydrogel solution with the cell suspension to form a soft hydrogel mixture;
adding additional cell culture medium to the soft hydrogel mixture to obtain cell colonies after a first time period;
harvesting the cell colonies;
mixing the hydrogel solution with the cell colonies to create a three-dimensional (3D) cell culture biomatrix;
adding cell differential medium to the hydrogel solution;
replacing, after a second time period, the cell differential medium with an organoid transfer medium; and
replacing, after a third time period, the organoid transfer medium with an organoid medium.

2. The method of claim 1, further comprising:
modifying the hydrogel solution with a functional ligand and/or a functional peptide,
wherein the functional ligand is selected from the group consisting of: RGD, a matrix metallopeptidase (MMP) sensitive ligand, a laminin functional ligand, a vitronectin functional ligand, a fibronectin functional ligand, an osteopontin functional ligand, a nidogen functional ligand, an elastin functional ligand, a thrombospondin functional ligand, and a collagen functional ligand,
wherein the functional peptide is selected from the group consisting of: a MMP functional peptide, a collagen functional peptide, a vitronectin functional peptide, laminin functional peptide, and a functional peptide molecule having an amine group, a carboxyl group, and an amide group, and
wherein the functional peptide molecule having an amine group, the carboxyl group, and the amide group is selected from the group consisting of: RGD, IKVAV, REDV, YIGSRY, poly Lysin.

3. The method of claim 1, further comprising:
adding an inhibitor/small molecule and/or a growth factor to the soft hydrogel mixture,
wherein the inhibitor/small molecule is selected from the group consisting of: ROCK1, Thiazovicin, CHIR99021, LY294002, A 83-01, Nicotinamide, SB 202190, Gastrin, DAPT, Forskolin, Prostaglandin E2, Testisteribe, SB 431542, retinoic acid, Y27632, MD2206, Dorsomorphin, G27632, and a smoothened agonist, and
wherein the growth factor is selected from the group consisting of: an epidermal growth factor (EGF), an Insulin-like growth factor (IGF), a fibroblast growth factor (FGF), R-Spondin, Wnt-3a, a bone morphogenetic protein (BMP), a hepatocyte growth factor, Activin A, a dickkopf-related protein, a brain-derived neurotrophic factor, a glial cell-derived neurotrophic factor, a sonic hedgehog, heregullin, prolactin, and Noggin.

4. The method of claim 1,
wherein the first time period is in a range of approximately one day to approximately seven days for growth of the cell colonies to become a size in a range of approximately 10 micrometers to approximately 1000 micrometers in diameter, and
wherein the second time period is in a range of approximately one day to approximately seven days.

5. The method of claim 1,
wherein an elastic modulus of the soft hydrogel mixture is in a range between approximately 0.01 Pa to approximately 2000 Pa, and
wherein the elastic modulus of the 3D cell culture biomatrix is in the range between approximately 1 Pa to approximately 5000 Pa.

6. The method of claim 1, further comprising:
adding an inhibitor/small molecule and/or a growth factor to the 3D cell culture biomatrix,
wherein the inhibitor/small molecule is selected from the group consisting of: ROCK1, Thiazovicin, CHIR99021, LY294002, A 83-01, Nicotinamide, SB 202190, Gastrin, DAPT, Forskolin, Prostaglandin E2, Testisteribe, SB 431542, Retinoic acid, Y27632, MD2206, Dorsomorphin, G27632, and a smoothened agonist, and
wherein the growth factor is selected from the group consisting of: an epidermal growth factor (EGF), an Insulin-like growth factor (IGF), a fibroblast growth factor (FGF), R-Spondin, Wnt-3a, a bone morphogenetic proteins (BMP), a hepatocyte growth factor, Activin A, a dickkopf-related protein, a brain-derived neurotrophic factor, a glial cell-derived neurotrophic factor, sonic hedgehog, heregullin, prolactin, and Noggin.

7. The method of claim 1, wherein the addition of the cell differential medium to the hydrogel solution is optional.

8. The method of claim 1,
wherein the addition of the cell differential medium to the hydrogel solution includes addition of the organoid transfer medium,
wherein the replacement of the cell differential medium with the organoid transfer medium after the second time period is omitted, and
wherein the replacement of the organoid transfer medium with the organoid medium after the third time period comprises replacement of the organoid transfer medium and the cell differential medium with the organoid medium after the second time period.

9. The method of claim 1,
wherein the replacement of the cell differential medium with the organoid transfer medium after the second time period comprises replacement the cell differential medium with the organoid transfer medium and the organoid medium after the second time period, and
wherein the replacement of the organoid transfer medium with the organoid medium after the third time period is omitted.

10. The method of claim 1,
wherein the cell culture medium includes a growth factor/protein and an inhibitor/small molecule to induce cells to the organoid culture,
wherein the growth factor/protein is selected from the group consisting of: an epidermal growth factor (EGF), an Insulin-like growth factor (IGF), a fibroblast growth factor (FGF), R-Spondin, Wnt-3a, a bone morphogenetic proteins (BMP), a hepatocyte growth factor, Activin A, a dickkopf-related protein, a brain-derived neurotrophic factor, a glial cell-derived neurotrophic factor, sonic hedgehog, heregullin, prolactin, and Noggin, and
wherein the inhibitor/small molecule is selected from the group consisting of: ROCK1, Thiazovicin, CHIR99021, LY294002, A 83-01, Nicotinamide, SB 202190, Gastrin, DAPT, Forskolin, Prostaglandin E2, Testisteribe, SB 431542, Retinoic acid, Y27632, MD2206, Dorsomorphin, G27632, and a smoothened agonist.

11. The method of claim 1, wherein cells of the cell suspension that are embedded or cultured in the 3D cell culture biomatrix are injectable for in vivo application.

12. The method of claim 1, wherein the cell suspension directly mixes with the hydrogel solution to create the 3D cell culture biomatrix and to induce cells for an organoid formation.

13. The method of claim 12, wherein the induction of the cells for the organoid formation occurs by:
adding the additional cell culture medium to the soft hydrogel mixture to form the cell colonies after a time period in a range of approximately one day to approximately fourteen days for growth of the cell colonies to become a size in a range of approximately 10 micrometers to approximately 1000 micrometer in diameter;
in response to the formation of the cell colonies, replacing the cell culture medium with the cell differential medium;
replacing, after the first time period in the range of approximately one day to approximately seven days, the cell differential medium with the organoid transfer medium; and
replacing, after the first time period, the organoid transfer medium with the organoid medium.

14. The method of claim 1, wherein the formation of the cell colonies is optional.

15. The method of claim 14, wherein cells can be induced directly for an organoid formation by adding the cell differential medium or the organoid medium.

16. A composition for a soft polysaccharide hydrogel capable of conversion to a hard polysaccharide hydrogel and suitable for injection uses, the soft polysaccharide hydrogel comprising:
one or more water soluble high acyl gellan gum polymers;
one or more water soluble low acyl gellan gum polymers; and
one or more water soluble chemically modified gellan gum polymers or one or more peptide modified gellan gum polymers, wherein the soft polysaccharide hydrogel exhibits a homogenous matrix structure and the hard polysaccharide hydrogel exhibits an aggregated matrix network structure.

17. The composition of claim 16,
wherein the soft polysaccharide hydrogel exhibits shear-thinning and self-healing rheological properties, by allowing the soft polysaccharide hydrogel to be converted into a free flowing (injectable) state by a shearing force, or to recover its hydrogel state once the shearing force is ceased, and
wherein the shearing force is exerted by a method selected from the group consisting of: pipetting, syringe injecting, and pump perfusion.

18. The composition of claim 16,
wherein the hard polysaccharide hydrogel exhibits 3D gel structures with rheological properties such that when the hard gel is broken by pipetting or shearing, the hard gel breaks into smaller gel particles, and has an affinity for one or more bioactive molecules or cells, and
wherein the hard polysaccharide hydrogel has a storage modulus value greater than approximately 10 Pa.

19. The composition of claim 18,
wherein each of one or more bioactive molecules are in contact with, adhered to, suspended in, entrapped in, or embedded in the soft polysaccharide hydrogel and the hard polysaccharide hydrogel while maintaining their bioactivities, and
wherein each of the one or more bioactive molecules release out from or move into the hydrogel.

20. The composition of claim 18, wherein the cells in the hydrogel maintain their bioactivities, grow in the hydrogel, or differentiate for functional cells or an organoid before or after in vivo injection.

21. The composition of claim 16, wherein the soft polysaccharide hydrogel is converted into the hard polysaccharide hydrogel by a method selected from the group consisting of: submersion in an aqueous solution of extra phosphate buffer, submersion in cell culture media, submersion in an ionic solution, and contact with bodily fluids (biofluids).
